(19) United States
(12) Patent Application Publication
(10) Pub. No.: US 2025/0336493 A1
(43) Pub. Date: Oct. 30, 2025

(54) METHODS AND SYSTEMS FOR PERSONAL DEVELOPMENT

(71) Applicants: Emadeldin El Sayed, Houston, TX (US); Adel El Sayed, Temple Terrace, FL (US)

(72) Inventors: Emadeldin El Sayed, Houston, TX (US); Adel El Sayed, Temple Terrace, FL (US)

(21) Appl. No.: 18/648,422

(22) Filed: Apr. 28, 2024

(51) Int. Cl. G16H 20/00; G06Q 50/00; G16H 10/20; G16H 80/00

(52) U.S. Cl. CPC G16H 20/00; G06Q 50/01; G16H 10/20; G16H 80/00

(57) ABSTRACT

A method for empowering a user, comprising the steps of establishing a plurality of pre-determined goals, storing a plurality of milestones related to each of the plurality of predetermined goals, having a user announce a stated goal, and determining whether the stated goal comprises one of the plurality of pre-determined goals. If the stated goal comprises one of the plurality of pre-determined goals, using the previously stored milestones. If the stated goal does not comprise one of the plurality of pre-determined goals, having the user establish a customized goal. If establishing a customized goal, the method comprises the steps of developing a plurality of customized milestones and developing a plurality of customized sub-milestones. The method further comprising the steps of having the user state a date of accomplishment and having the user state an affirmation statement.

### PRIORITY CLAIM

This non-provisional patent application claims the benefit of U.S. Provisional Application No. 63/472,206 filed on Jun. 9, 2023, the entirety of which is incorporated herein by reference.

### FIELD

The present disclosure generally relates to personal development. More specifically, the present disclosure relates to apparatus, methods, and systems for personal development. Particularly, the disclosure is related to an interactive personal development mobile application that provides access to motivational and goal-oriented tools to assist individuals to strive towards their highest potential and achieve a desired and targeted goal.

### BACKGROUND

There are many reasons why people fail to achieve their stated goals and desires. For example, some individuals fail to set or articulate a specific goal. If someone does not know what they want to achieve, it is much harder to actually achieve it. Other individuals fail to achieve their stated goals or desires since they fail to implement an action plan. Once they know what they want to achieve, they need to create a plan for how they are going to progress to their targeted goals. This plan should include specific steps, timelines, and resources.

Others fail to achieve their stated goals and desires since they do not take action. Having a plan is great, but it is not enough. Individuals need to take action towards the stated goals. This means taking steps repeatedly, every day, even if these are small steps. Other individuals fail to achieve their goals since individuals must be persistent in striving to achieve their goal. There will be times when individuals want to give up on a stated goal. But if individuals want to achieve a stated goal, they need to be persistent. And others fail to achieve their goals because they simply fail to receive reminders. If not reminded continuously and often, it is easy for individuals to forget the stated goal or desire.

Therefore, what is generally needed is a platform to assist individuals unleash their potential and reach their goals through positive thinking, self-motivation, and one or more available resources. Platforms are required that allow individuals to achieve their goals while connecting others across the platform and across the world. What is also needed is an automatic system or mobile application that simplifies and makes more efficient self-improvement, self-care, personal development, and productivity mobile application that certain individuals having a certain religious faith (e.g., Muslims) needs, or other users from different faith background, for personal growth and goal achievement; personal, educational, career, financial and other goals. There is also a general need for a system or mechanism that allows for goals to be stated, affirmed, supported, and nurtured through various aspects while at the same time improving overall wellness, mental health, and/or the mindset of the individual seeking his or her goal.

### SUMMARY

According to an exemplary arrangement, a method for empowering a user, comprising the steps of establishing a plurality of pre-determined goals; storing a plurality of milestones related to each of the plurality of predetermined goals; having a user announce a stated goal; and determining whether the stated goal comprises one of the plurality of pre-determined goals. If the stated goal comprises one of the plurality of pre-determined goals, using the previously stored milestones. If the stated goal does not comprise one of the plurality of pre-determined goals, having the user establish a customized goal. If establishing a customized goal, the method comprises the steps of developing a plurality of customized milestones and developing a plurality of customized sub-milestones. The method further comprises the steps of having the user state a date of accomplishment and having the user state an affirmation statement.

In one arrangement, the method further comprising the step of having the user state an importance of the stated goal.

In one arrangement, the method further comprising the step of generating a reminder image that operates to remind the user of the stated goal.

In one arrangement, the method further comprising the step of performing an audio affirmation of recording the users stating an affirmation statement.

In one arrangement, the method further comprising the step of uploading an affirmation audio file.

In one arrangement, the method further comprising the step of selecting whether the stated goal will comprise a public stated goal or a private stated goal.

In one arrangement, the method further comprising the step of allowing the stated goal and the user to be searchable.

In one arrangement, the method further comprising the step of generating a motivation score.

In one arrangement, the motivation score comprises a purpose score, a wellness score, and a goal score.

In one arrangement, the motivation score is based in part on a user self-assessment questionnaire.

In one arrangement, the method further comprising the step of providing a recommendation list to the user based in part on the motivation score.

In one arrangement, the method further comprising the step of providing a plurality of sorted publicly available articles based in part on the motivation score.

In one arrangement, the method further comprising the step of providing a plurality of sorted publicly available videos and articles-based criteria underlying the computed motivation score.

In one arrangement, the method the recommendation list comprises a plurality of recommended articles.

In one arrangement, the method further comprising the step of performing an affirmation exercise.

In one arrangement, the method further comprising the step of performing visualization exercises.

In one arrangement, the method further comprising the step of suggesting articles for assisting the user to achieve the stated goal.

In one arrangement, the method further comprising the step of assisting the user with learning about developing a plan and achieving the stated goal.

In one arrangement, the method further comprising the step of providing a plurality of readable affirmations.

In one arrangement, the method further comprising the step of providing access to affirmations and visualizations.

In one arrangement, the method further comprising the step of repeatedly playing the user's affirmations.

In one arrangement, the method further comprising the step of providing extra affirmations.

In one arrangement, the method further comprising the step of providing a plurality of motivational tools.

In one arrangement, the motivational tools are selected from the group comprising motivational quotes and inspirational quotes.

In one arrangement, the method further comprising the step of providing a social platform for exchanging ideas.

In one arrangement, the method further comprising the step of establishing a chat platform for exchanging communications.

In one arrangement, the method further comprising the step of creating notifications to be sent to the user regarding the stated goal.

In one arrangement, the method further comprising the step of generating electronic coaching, E-Coaching, statements based on a user's motivation score.

In one arrangement, the method further comprising the step of prompting the user to say a prayer about the stated goal.

The features, functions, and advantages can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 illustrates an exemplary graphical user interface (GUI) for use with a mobile application for use with a mobile computing device, like a smartphone, a tablet, a professional digital assistant or other similar like device. FIG. 1 illustrates the goal development screen where the user entry key goal entries; name of the goal, category, affirmation statement, dates of accomplishment, why this goal is important, picture of the goal, audio affirmation, choice to make the goal public or private, and whether wants to get suggested articles to get educated on the goal and how to make a goal plan. In the case of the pre-determined goals, some fields are pre-populated such as; goal name, goal category, and goal picture.

This goal setting feature can be important for a number of reasons. For example, it allows users to cultivate their vision for success by developing specific, measurable, achievable, relevant, and time-bound (SMART) goals with an Islamic approach. In addition, users will understand their intentions and reasons for setting goals and while keeping their motivation strong.

In addition, user will be able to utilize the systems comprehensive goal tracker to manage all of their goals in one place, allowing users to monitor progress and stay on track. Users will also be able to access voice affirmations for daily meditation and mindfulness to reinforce positive thinking and self-belief.

FIG. 2 illustrates another exemplary GUI related to a goal picture to help the user visualize achieving the stated goal. In the case of pre-determined goals, the goal picture is pre-selected but can be edited by the user. In the case of new customized goals, the user has to upload a goal picture from their phone.

FIG. 3 illustrates an exemplary GUI related to an audio affirmation screen where the user records the audio affirmation. It also shows the user the guidance on how to do a daily affirmation.

FIG. 4 illustrates an exemplary GUI related to an audio affirmation screen where the user uploads an audio file from their mobile device.

FIG. 5 illustrates an exemplary GUI related to a goal development screen where the user chooses to make the goal public or private, and whether to select the suggested articles to get educated on the goal and how to make a goal plan.

FIG. 6 illustrates an exemplary GUI where the user selects a category from the predetermined goals categories then selects a certain goal from each category.

FIG. 7 illustrates an exemplary GUI related to a goal plan template with goal milestone, goal connection to assist with the goal plan, and whether it is completed or not.

FIG. 8 illustrates an exemplary GUI related to a goal plan edit screen to give the user the ability to pick day of the week, frequencies of how many times per day to perform the task, and duration.

FIG. 9 illustrates an exemplary GUI related to a user's today action and milestone to complete, and how much it contributes to the overall goal plan progress. It also shows the plan resources to educate the user on how to develop a goal plan for the selected goal.

FIG. 10 illustrates an exemplary GUI related to a motivation score assessment screens for Goal. The user answers these questions in the beginning to form a baseline score, then continually take the re-assessment. It also suggests the user to upgrade to membership to unlock the motivation score feature.

FIG. 11 illustrates an exemplary GUI related to motivation score assessment screens for goal and its category definition, purpose and its definition, and wellness and its definition. The user answers these questions in the beginning to form a baseline score, then continually takes the re-assessment. It also provides suggestions to improve the goal score definition.

With the presently disclosed systems and methods, users will be able to assess their motivation levels in real-time with our unique motivation score. Importantly, some of these features were developed by psychiatrists and coaches, wherein this assessment will help users identify areas where they may need to work on and provides personalized recommendations to boost motivation.

FIG. 12 illustrates an exemplary GUI related to a screen to ask the user for their preference on how they want to hear their affirmation; a) audio affirmation only b) music audio or c) audio affirmation with the music audio in the background. It also provides the user with the guidance on how to say their daily affirmations. It also shows the user tracking on the suggested number of affirmations (14 times per day).

Positive affirmations and empower quotes can be important for a number of reasons. The presently disclosed systems and methods will provide users with access to an extensive collection of hundreds of positive affirmations, empowering quotes, including Islamic words and Qur'anic verses. The presently disclosed systems and methods will allow users to browse through different categories to find inspiration and increase your self-belief and self-confidence in achieving your goals.

FIG. 13 illustrates an exemplary GUI related to how to add a goal connection to your goal plan milestones, sharing the stated goal or goal plan with others, and listing top three suggested articles for goal resources and plan resources.

FIG. 14 illustrates an exemplary GUI related to displaying the affirmation categories, how to say affirmations, sharing selected affirmations with other users, suggesting the user to say God Willing/Insha'Allah after each affirmation, and sliding right or left to read more.

FIG. 15 illustrates an exemplary GUI related to displaying the famous quotes categories, sharing selected quotes with other users, and sliding right or left to read more.

FIG. 16 illustrates an exemplary GUI related to displaying the Empower categories to play empowerment videos and read empowerment articles that are publicly available but vetted and sorted out in the same categories as the motivation score.

FIG. 17 illustrates an exemplary GUI related to chatting with goal connections to leverage best practices and exchange ideas on the stated goals.

FIG. 18 illustrates an exemplary GUI related to notification settings for E-Coaching, Prayer/Dua Reminder, Affirmation Reminder, Goal Plan, Motivation Quotes, and Extra Affirmation. The user will also be able to select “Helpful” or “Not Helpful” for the different notifications.

FIG. 19 illustrates an exemplary GUI related to E-Coaching push notifications that are generated based on the user motivation scores in the different categories. The presently disclosed systems and methods can serve as a user's dedicated accountability coach. In this manner, users can receive personalized coaching recommendations throughout the day based on the goal motivation score, ensuring that users stay motivated and focused on the path to achievement. Users will therefor benefit from expert guidance and support until they successfully attain their goals, empowering them to move forward to the next phase of self-improvement.

FIG. 20 illustrates an exemplary GUI related to Muslim prayer/Dua reminder for the user to say to improve their self-beliefs and motivations toward the state goals.

FIG. 21 illustrates an exemplary process flow diagram for use with a mobile application for use with a mobile computing device, like a smartphone, a tablet, a professional digital assistant or other similar like device.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

In one arrangement, the present disclosure is related to systems and methods such as a mobile application for use with a computing device, such as a handheld computing device. Such a handheld computing device may comprise a smart phone, a tablet, a laptop computer, or other similar compute device. The mobile application can be directed to certain goal setting, goal planner, motivation, and the Muslim positive affirmations. As described in detail herein, the presently disclosed mobile application comprises a Muslim based mobile application that integrates certain necessary elements to empower a user in actually achieving his or her stated goal or goals.

The presently disclosed mobile application, in one preferred arrangement, provides a platform to help Muslims unleash their potential and reach their goals through positive thinking, self-motivation, and one or more available resources. This can be achieved while connecting their powerful minds across the platform and across the world. More specifically, the presently disclosed mobile platform comprises a self-improvement, self-care, personal development, and productivity mobile application that certain individuals having a certain religious faith (e.g., Muslims) needs, or other users from different faith background, for personal growth and goal achievement; personal, educational, career, financial and other goals. These goals can be stated, affirmed, supported, and nurtured through various aspects of the mobile application while at the same time or simultaneously improving overall wellness, mental health, and/or the mindset of the mobile application user.

The presently disclosed mobile app helps the users to identify and clarify their purpose by asking them certain questions (e.g., utilizing certified coaching questions), establishing goals that align with their purpose, and developing detailed goal plans to achieve their goals. At the same time or concurrently, the mobile application also provides various resources that align or support the user's efforts in achieving his or her proclaimed goal.

In one preferred arrangement, once the users get a better understanding of and clarity on their purpose, their goals which are aligned to such purpose would be easier to pursue, driven by the user intrinsic motivators. That is, these user intrinsic motivators concern why the users are purposing such goals.

From a goal plan development perspective, at least in one preferred arrangement, the app helps the users develop a straightforward goal plan then it continues to be refined and detailed until the user achieves their goals. To increase the user's overall motivation about pursuing these stated goals, the mobile application assesses the user's efforts and disposition based on several key metrics or goal parameters. As just one example, these key metrics may concern the user's purpose, the user's health, and/or the user's goals. Once the application assesses the user based on these one or multiple key metrics, then the application preferably integrates these key features so as to strengthen the user self-belief and confidence. In one preferred arrangement, the user's self-belief and confidence is strengthened by using certain proven psychological science and project management tools.

In one preferred arrangement, the mobile application provides a list of features and integrates these features so as to help users achieve their stated goals and improve their overall motivation. In one preferred arrangement, the key features may comprise at least one or more of the following: goal development, goal planning, motivation score, affirmation and visualization exercises, suggested third party sources or published articles, readable affirmations, famous quotations, empowerment videos and articles, chats, push notifications, electronic or E-Coaching statements, and saying prayers for their goals. One or more aspects regarding the presently disclosed mobile application is scientifically proven to work. It has a plurality of platform features that will help the user accomplish their goals while developing a positive outlook on life and it empowers the user through its key features. A number of these key features are summarized below.

The presently disclosed mobile app helps users cultivate their vision for success by developing specific, measurable, achievable, relevant, and time-bound (SMART) goals. In one preferred arrangement the presently disclosed mobile app helps users cultivate their vision for success supported by Islamic principles.

Utilizing aspects of the presently disclosed mobile application helps users understand their intentions and reasons for setting goals. At the same time, the presently disclosed mobile application also keeps user motivation strong. This can be achieved via multiple motivators such as with the help of online resources on Islamic productivity and self-improvement. In one preferred arrangement, users can utilize the mobile app's goal tracker to manage all personal and professional goals in one place and can also be utilized to access voice affirmations periodically, such as for daily meditation and mindfulness.

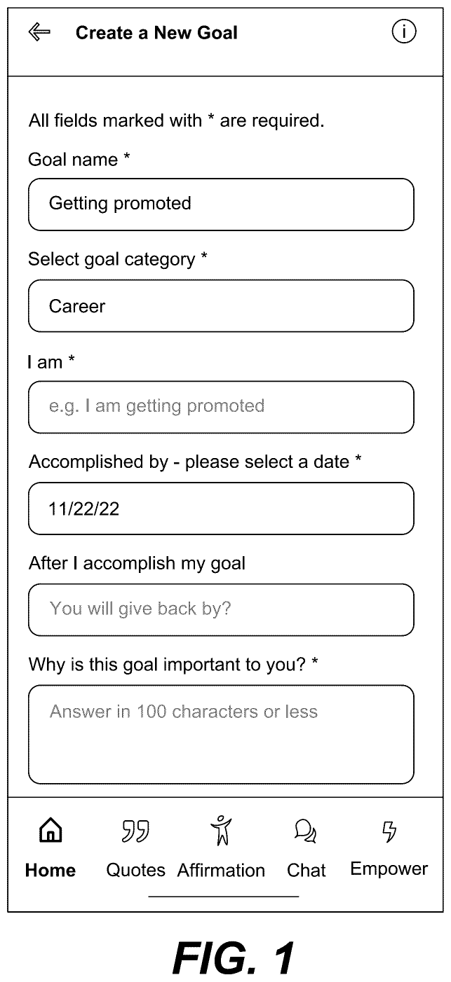

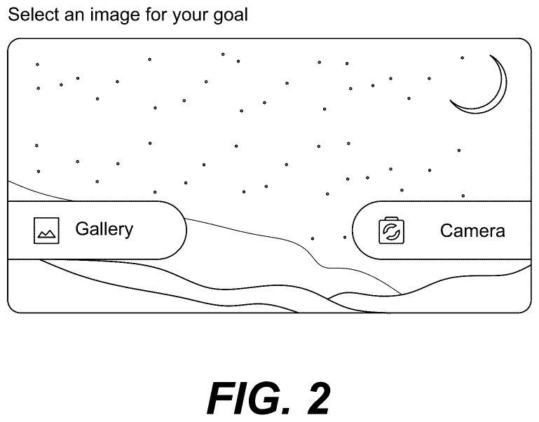

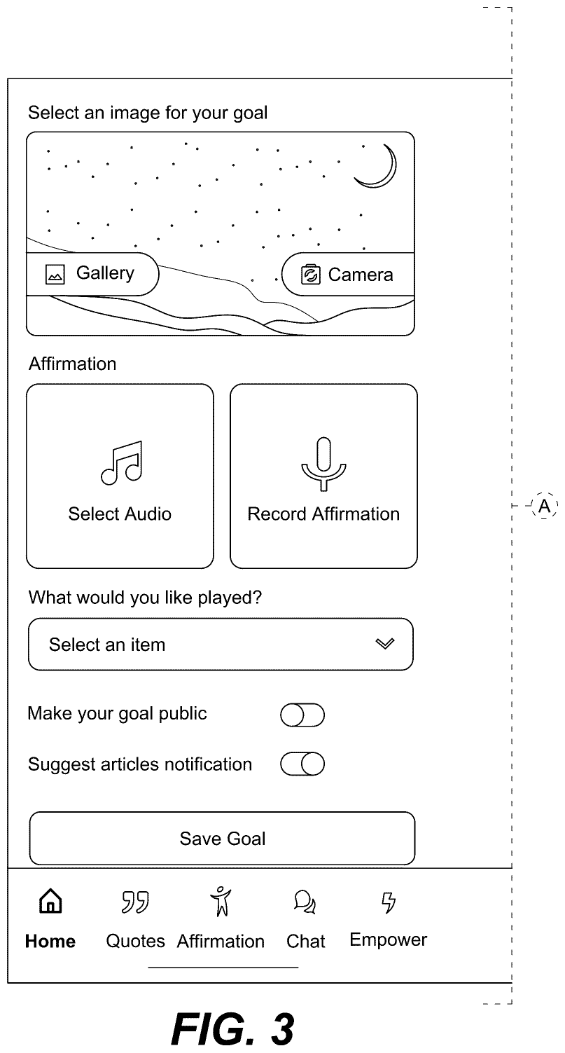

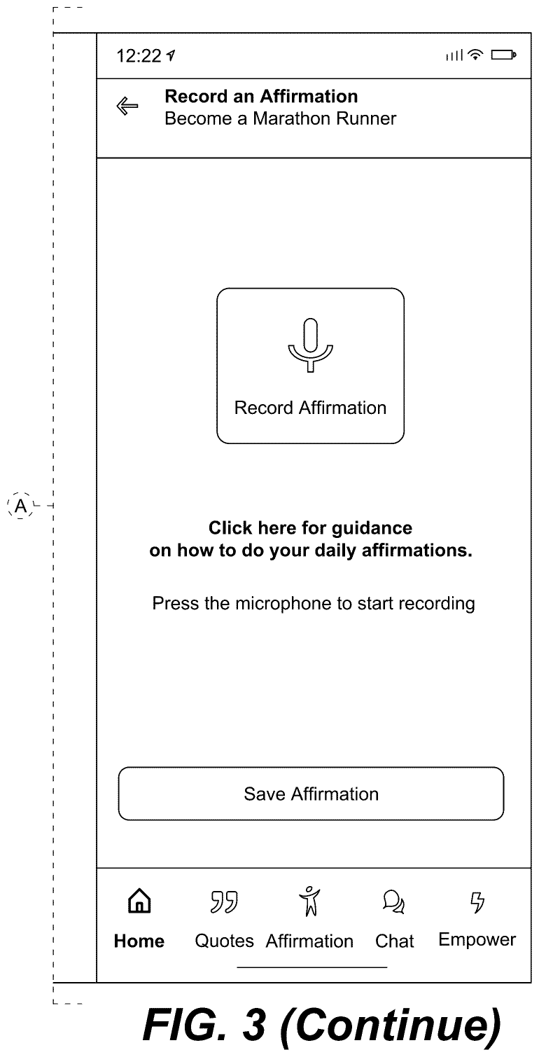

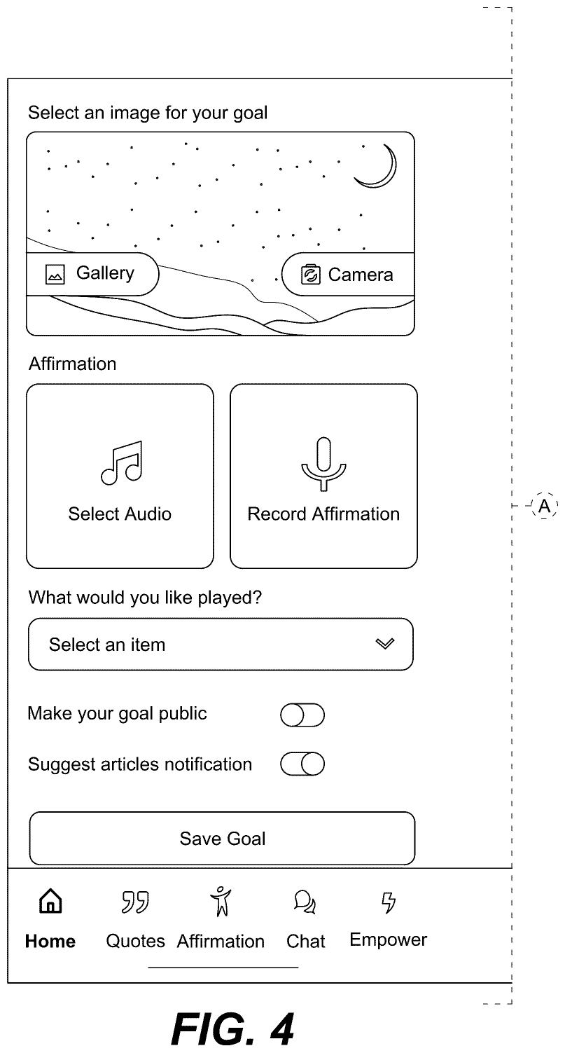

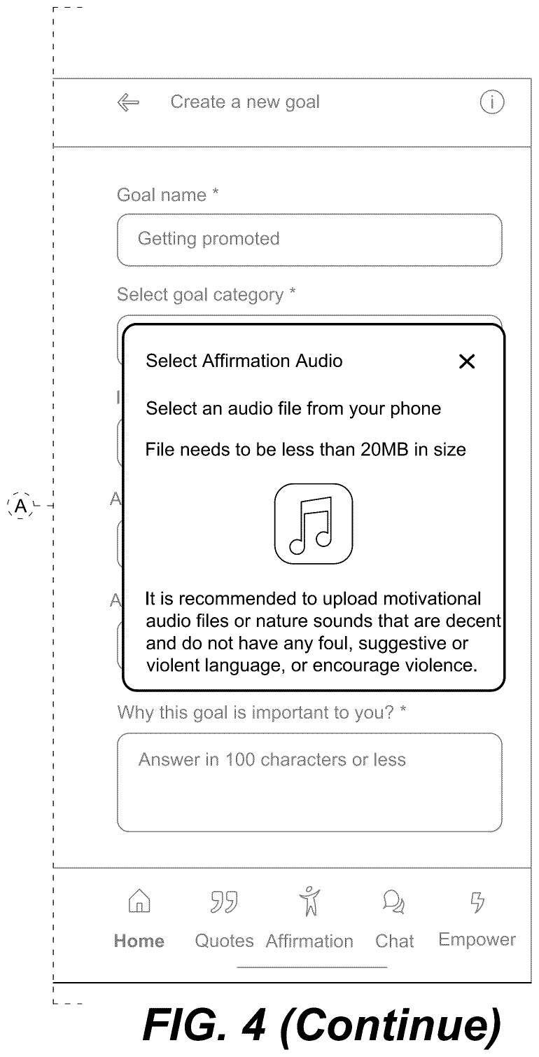

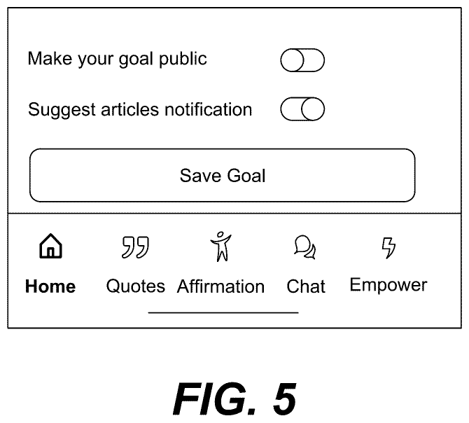

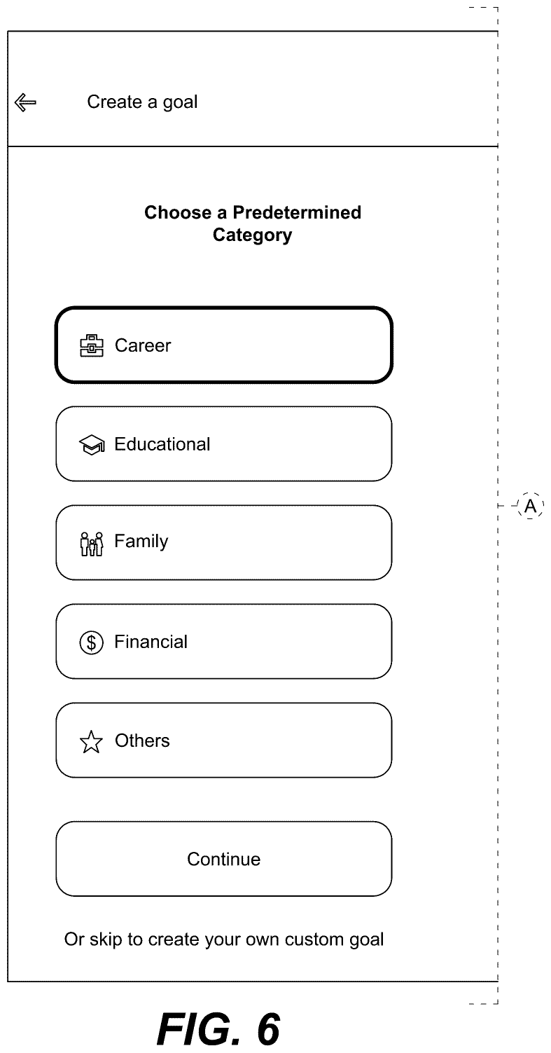

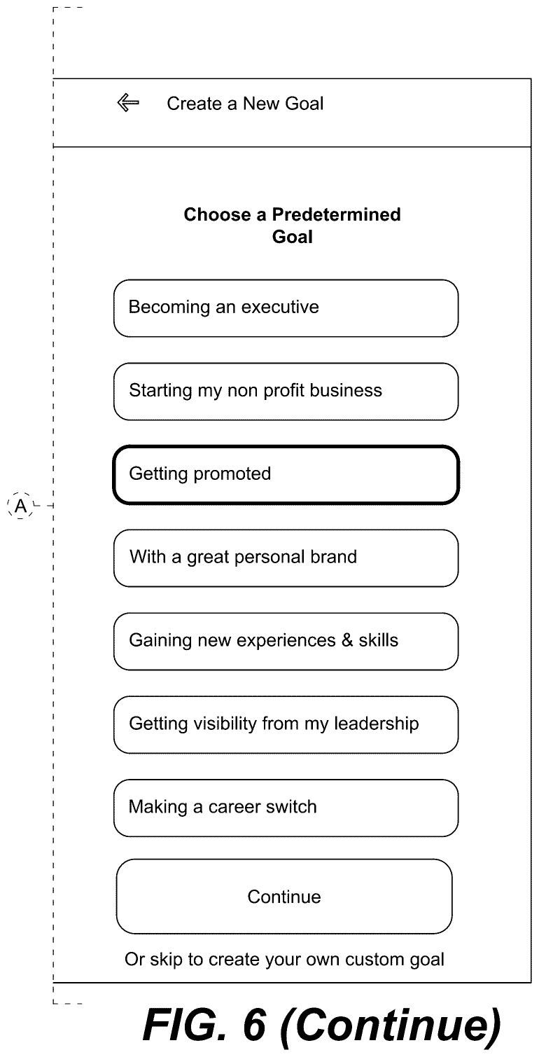

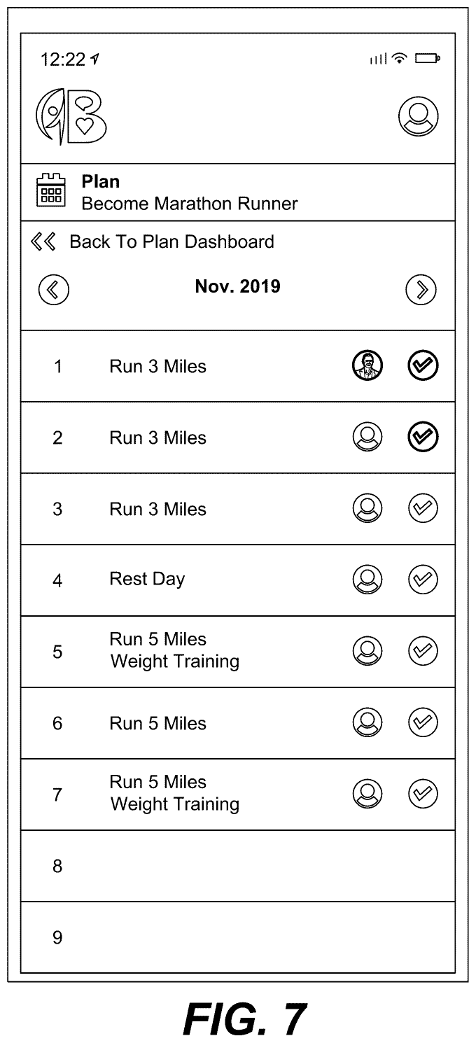

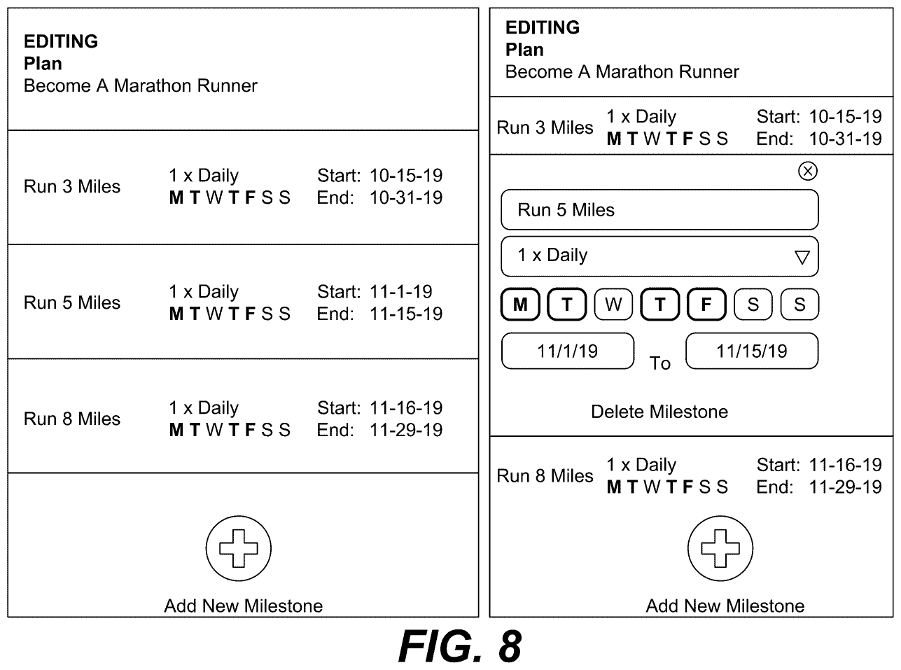

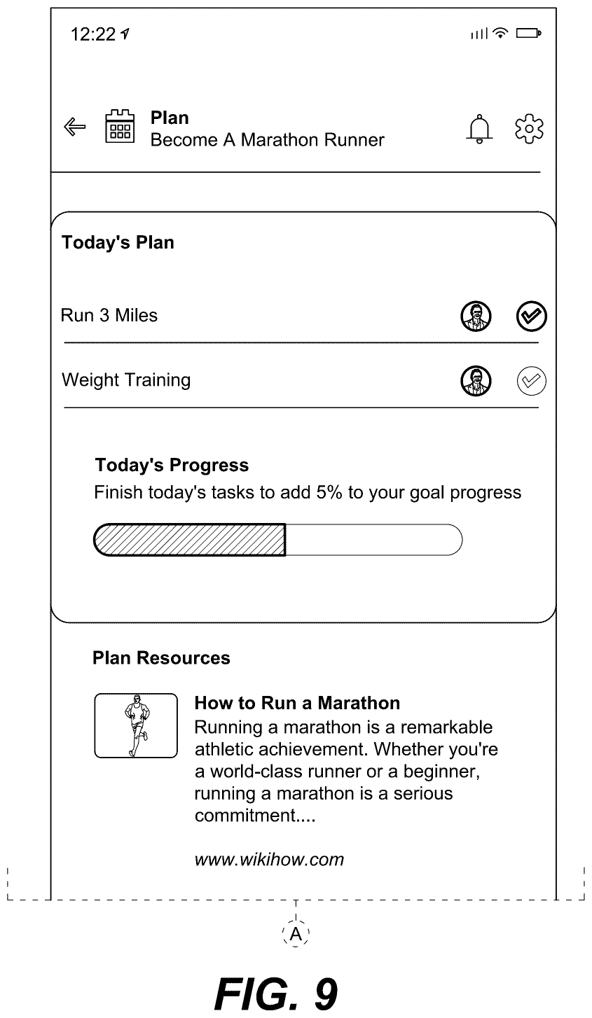

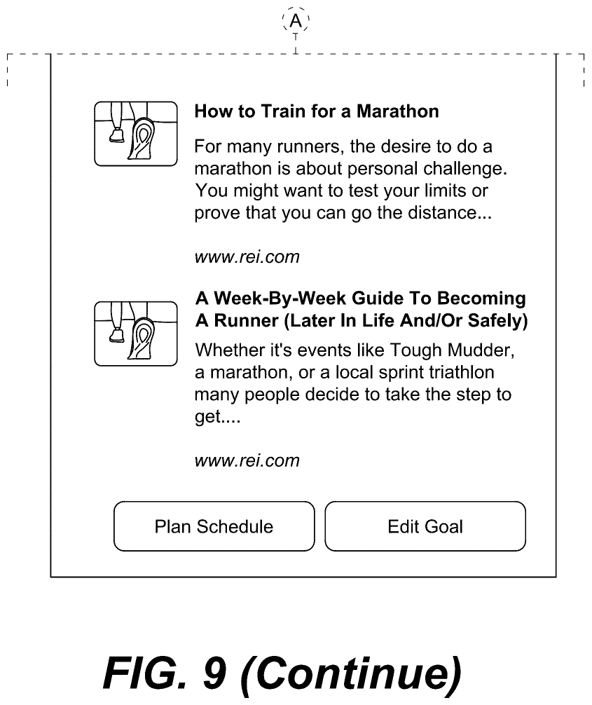

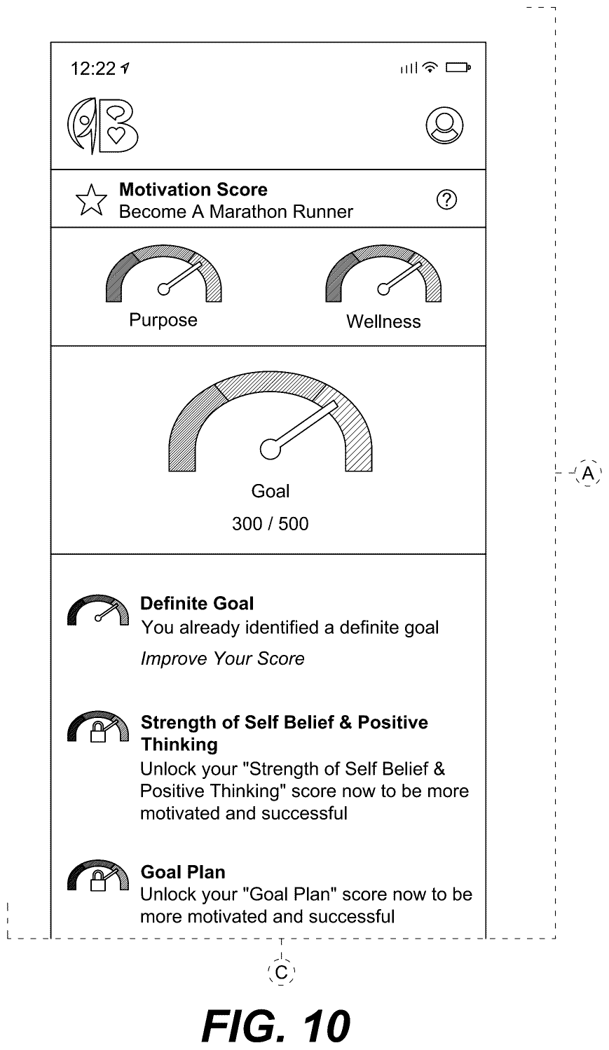

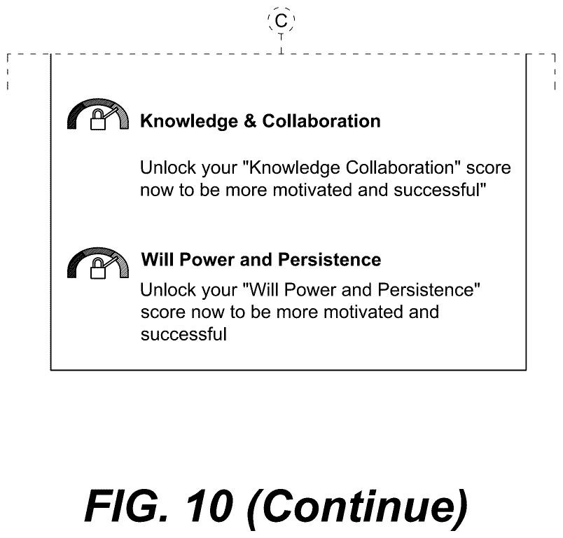

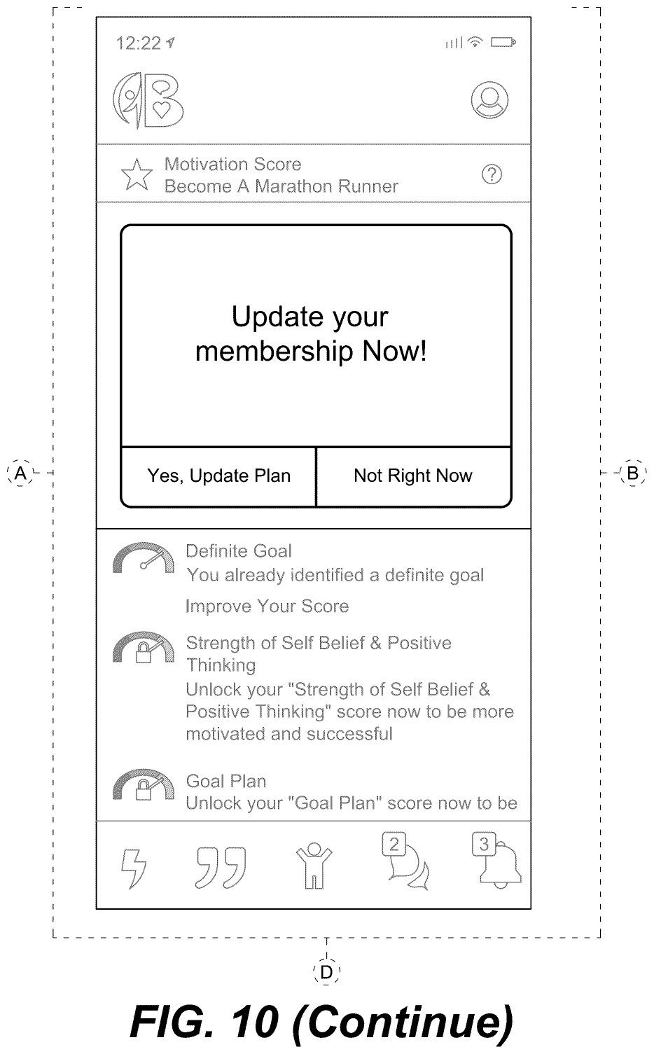

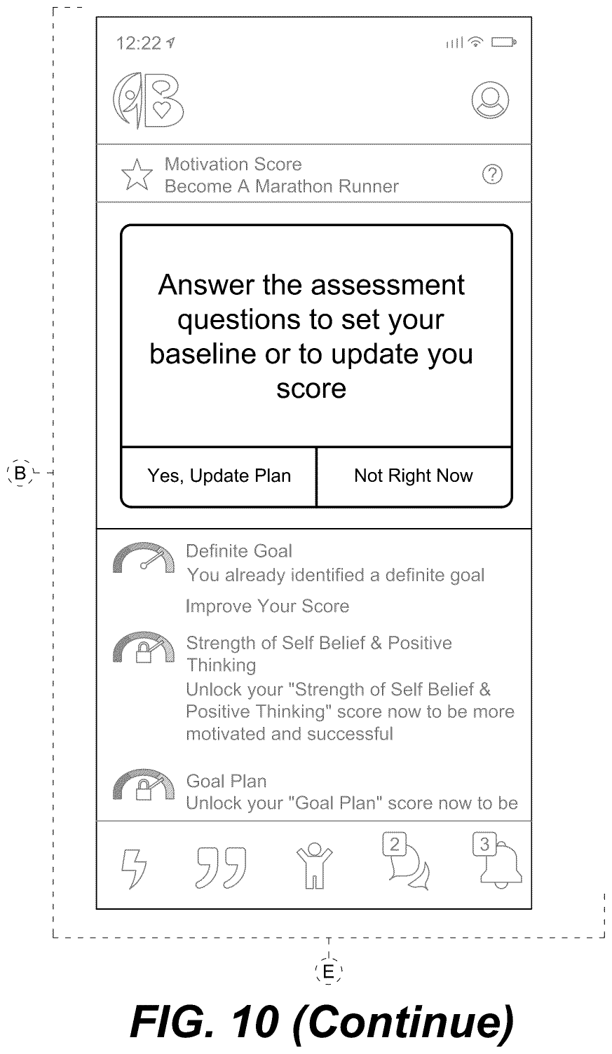

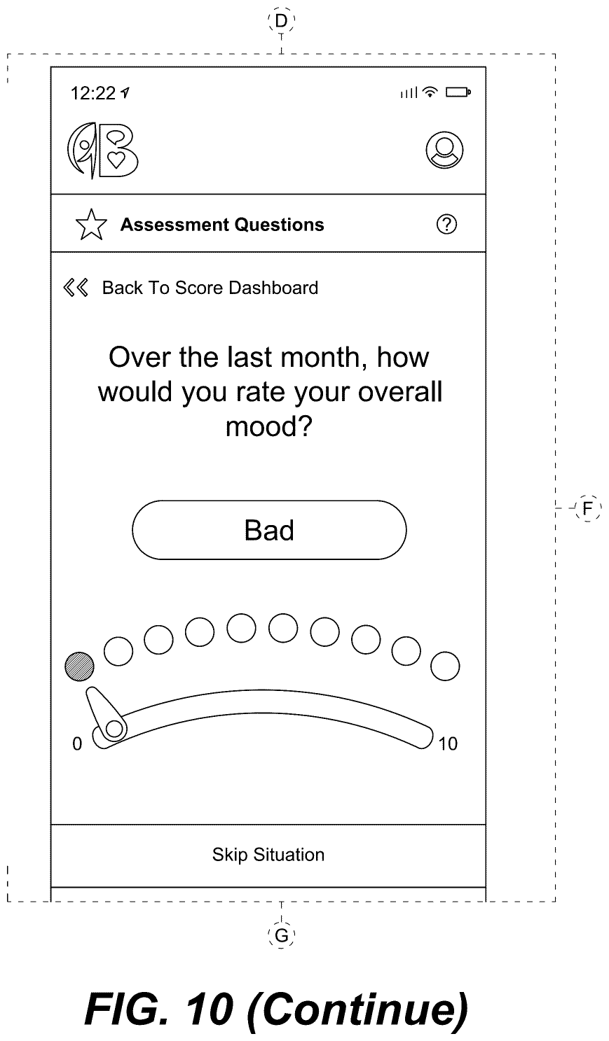

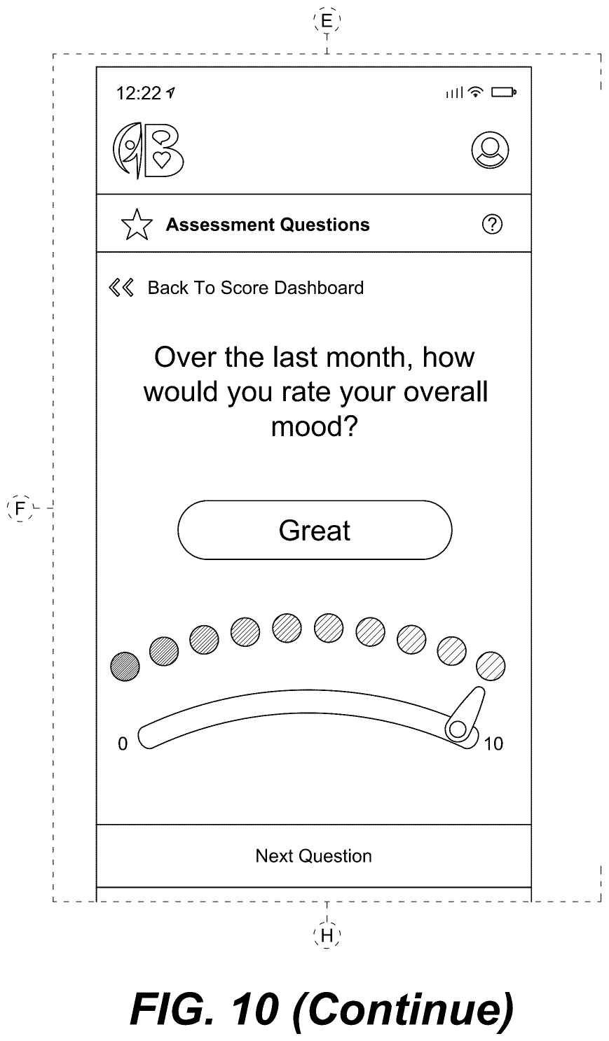

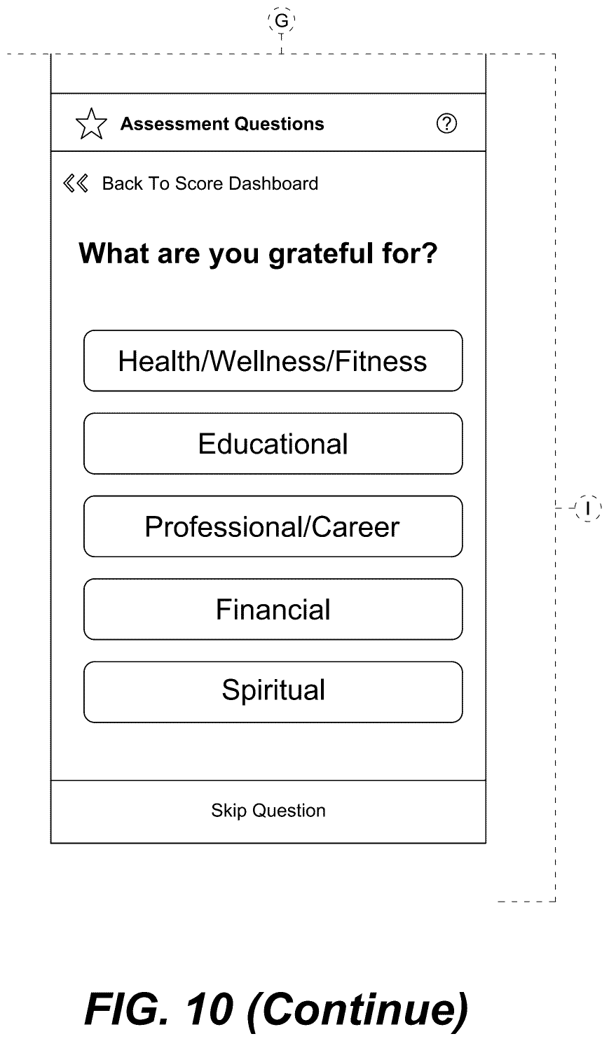

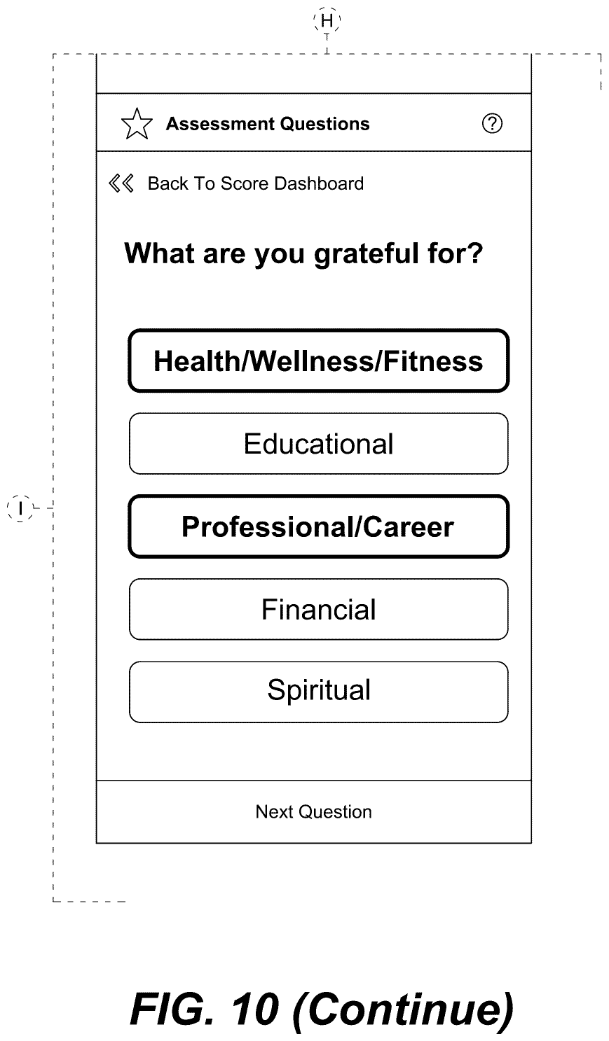

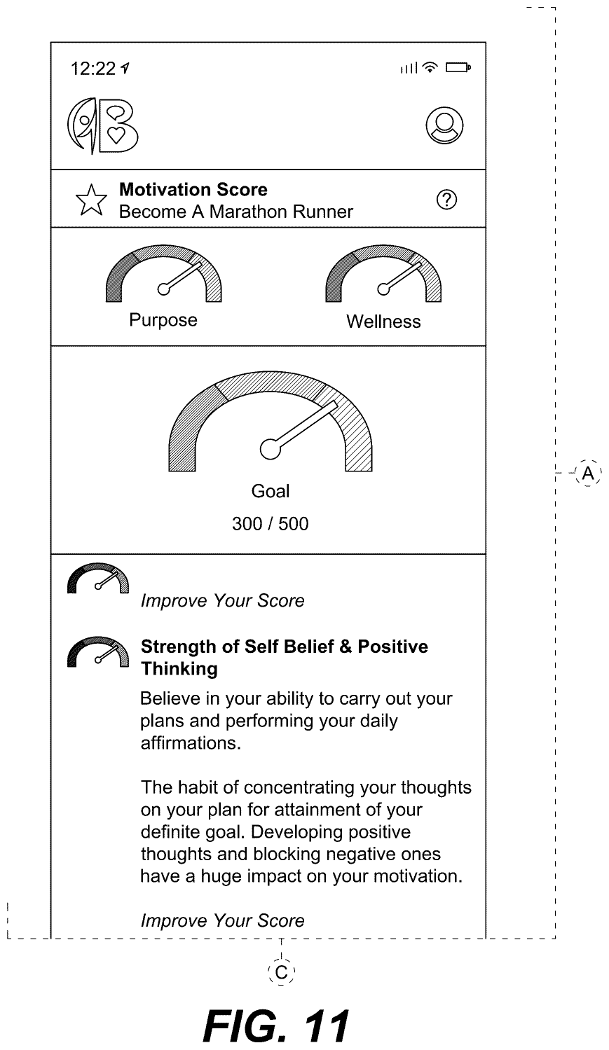

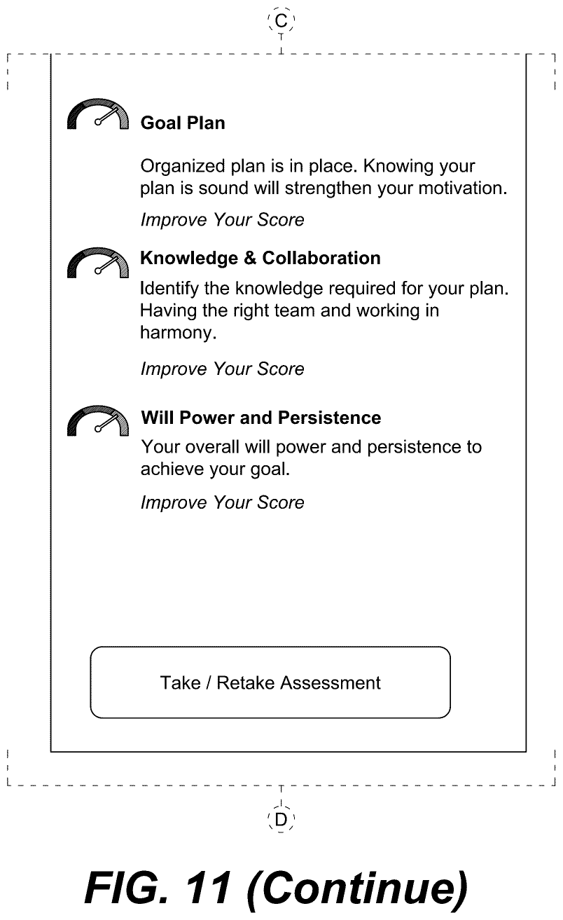

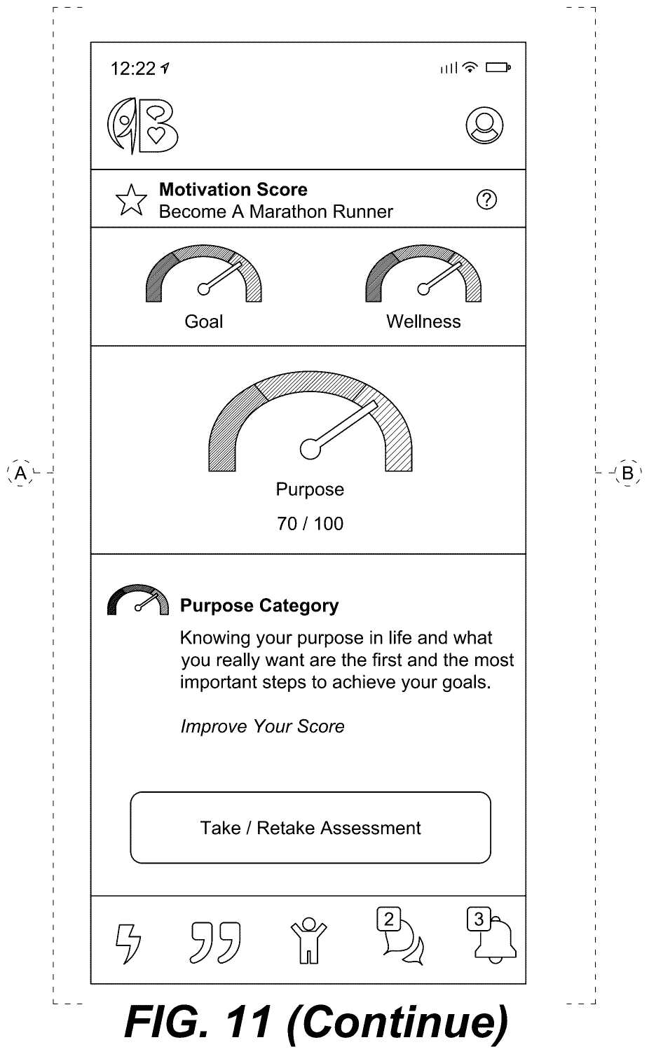

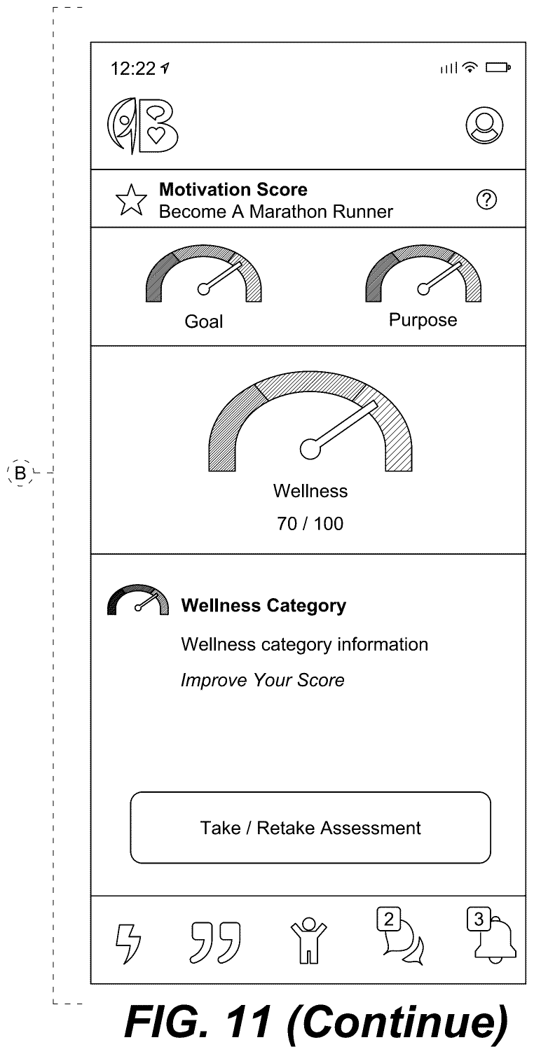

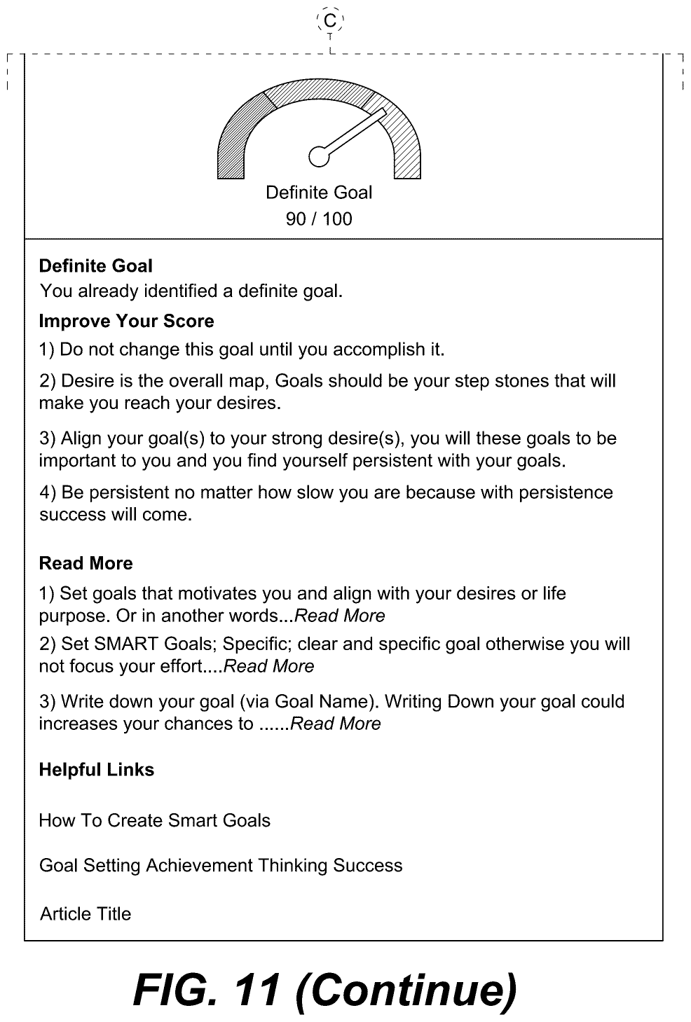

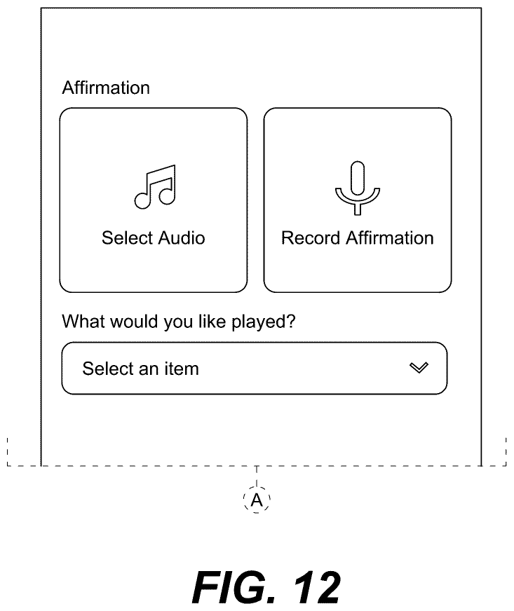

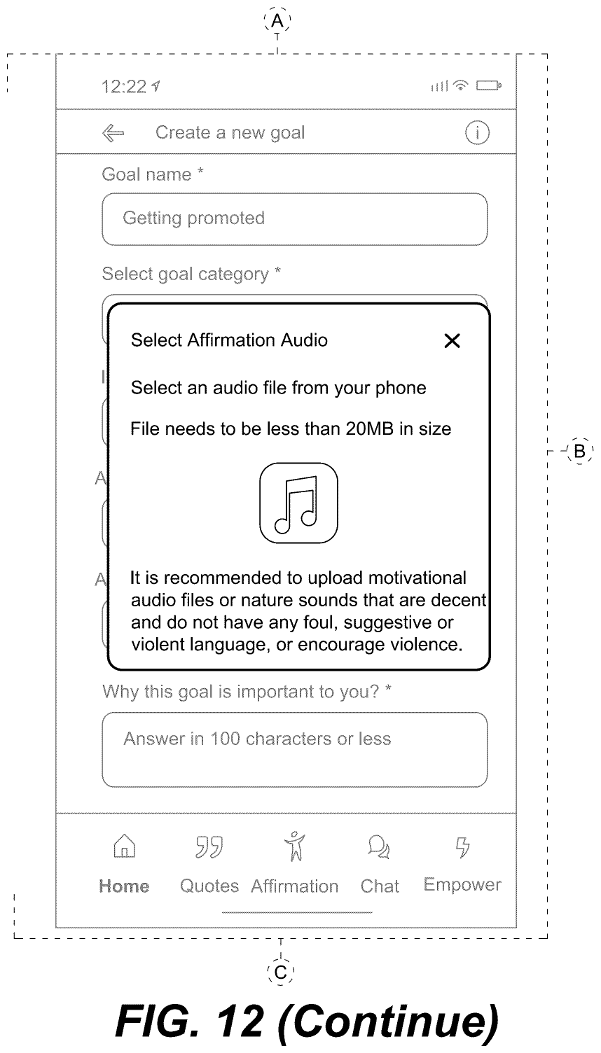

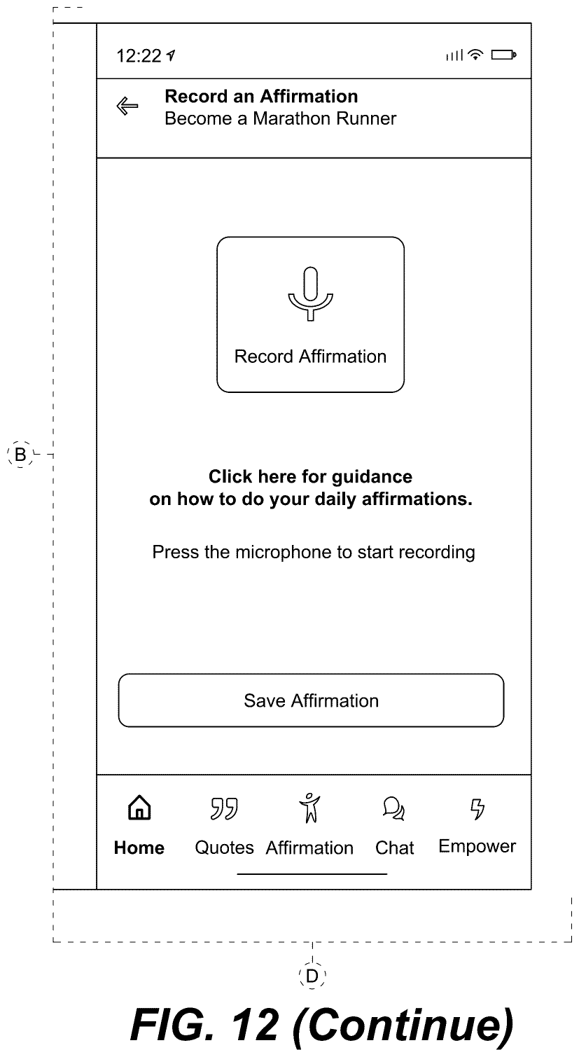

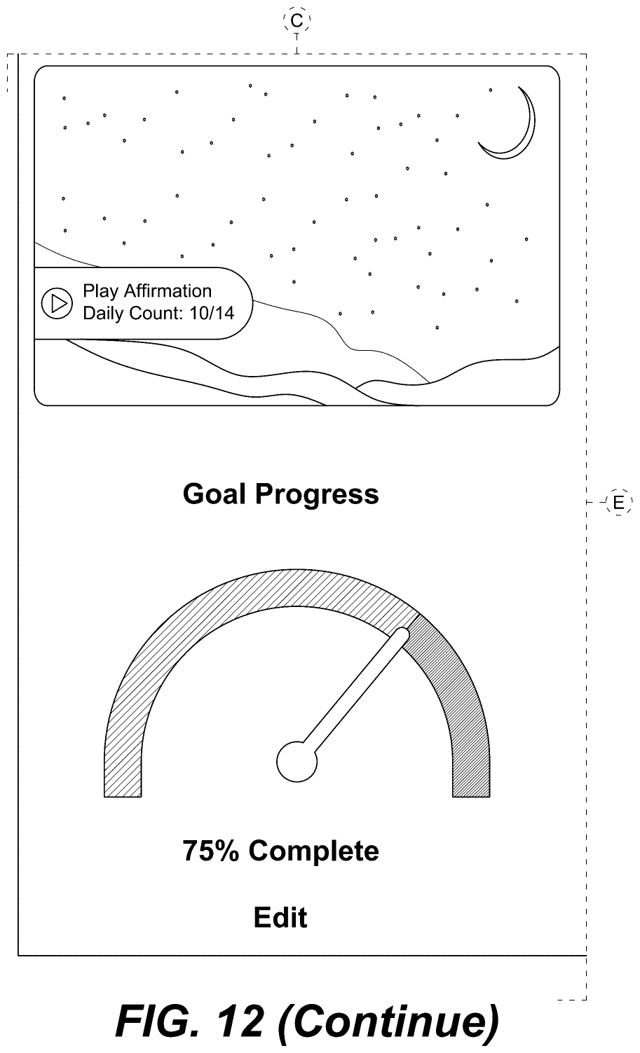

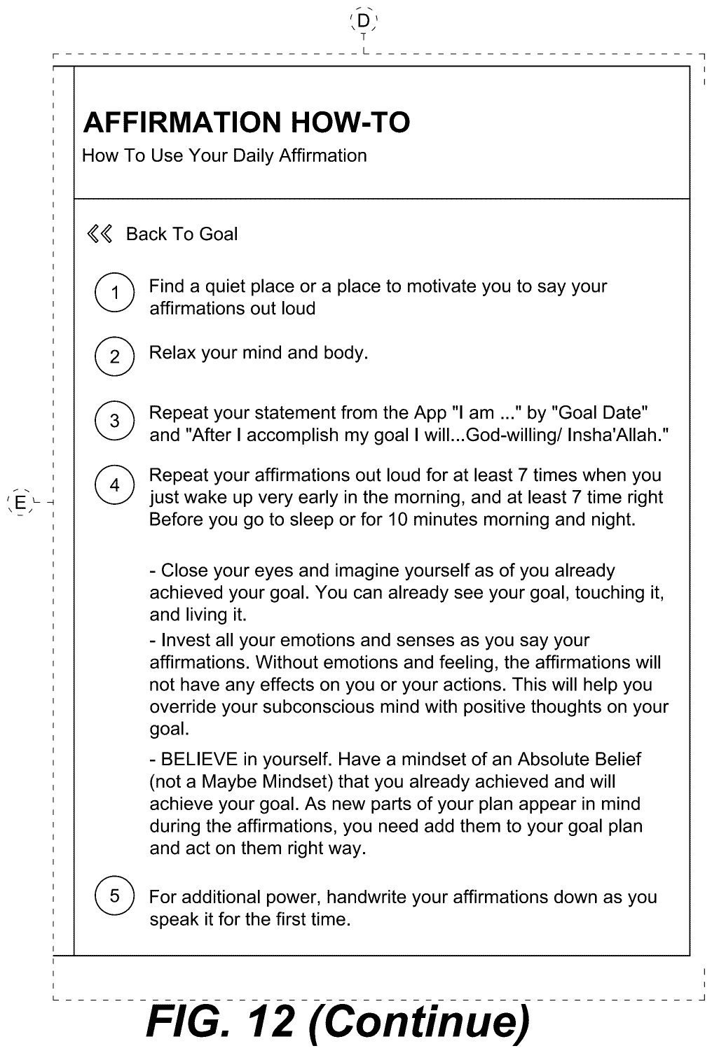

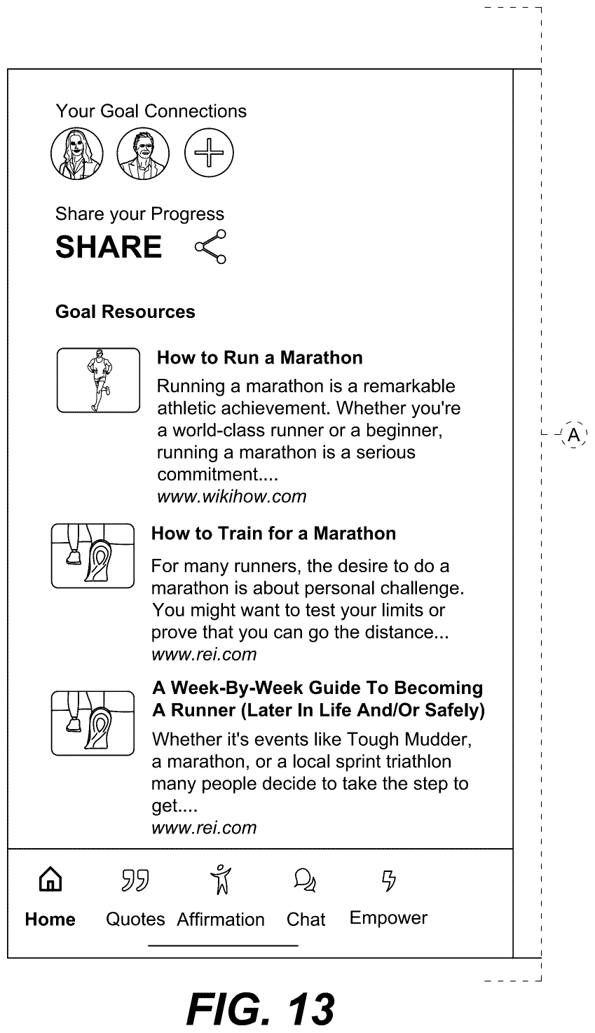

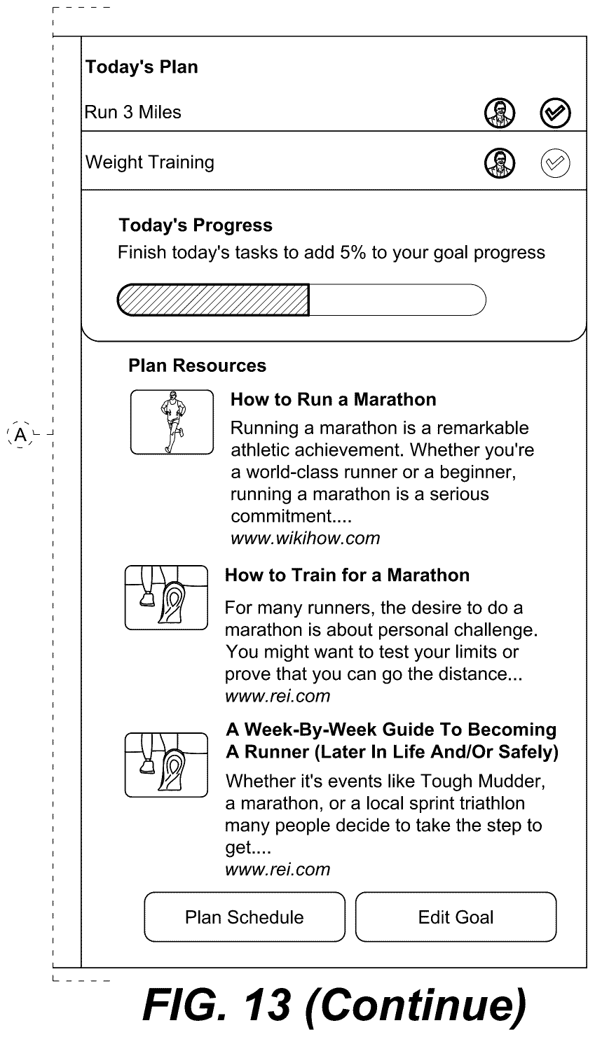

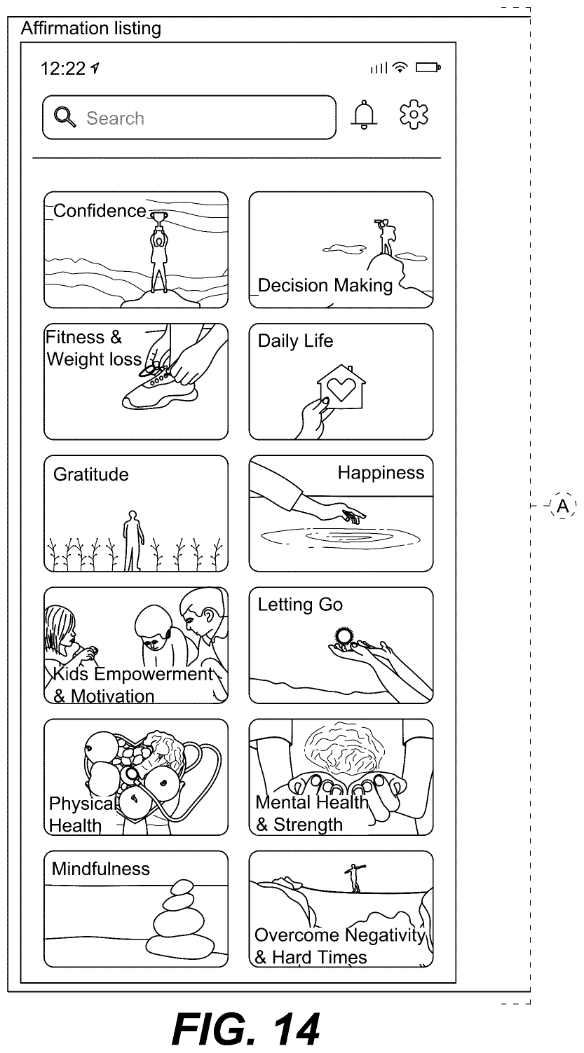

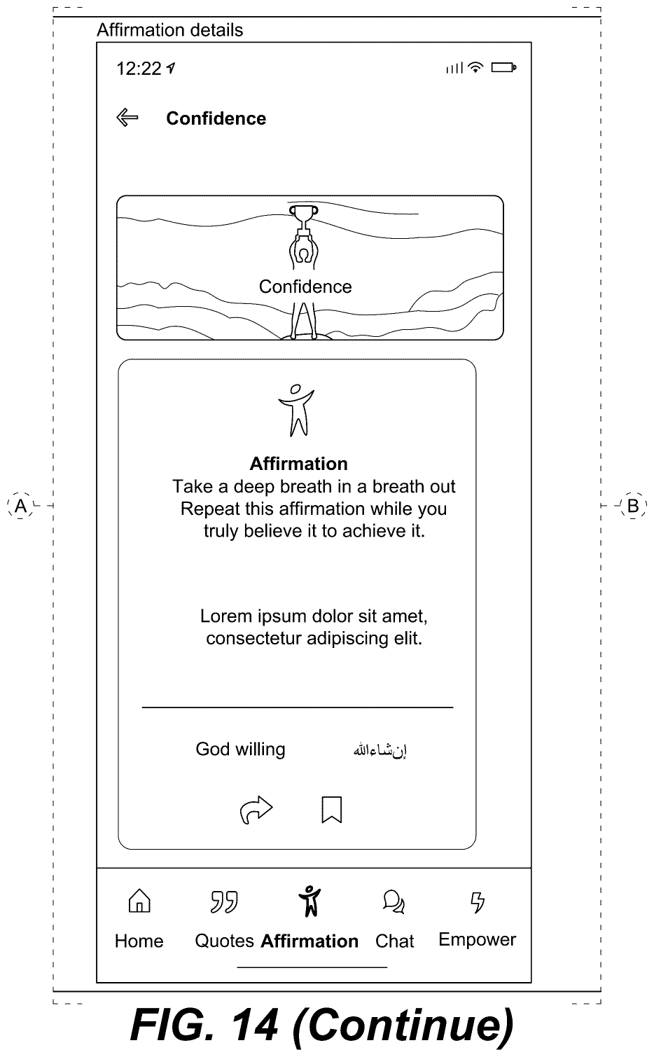

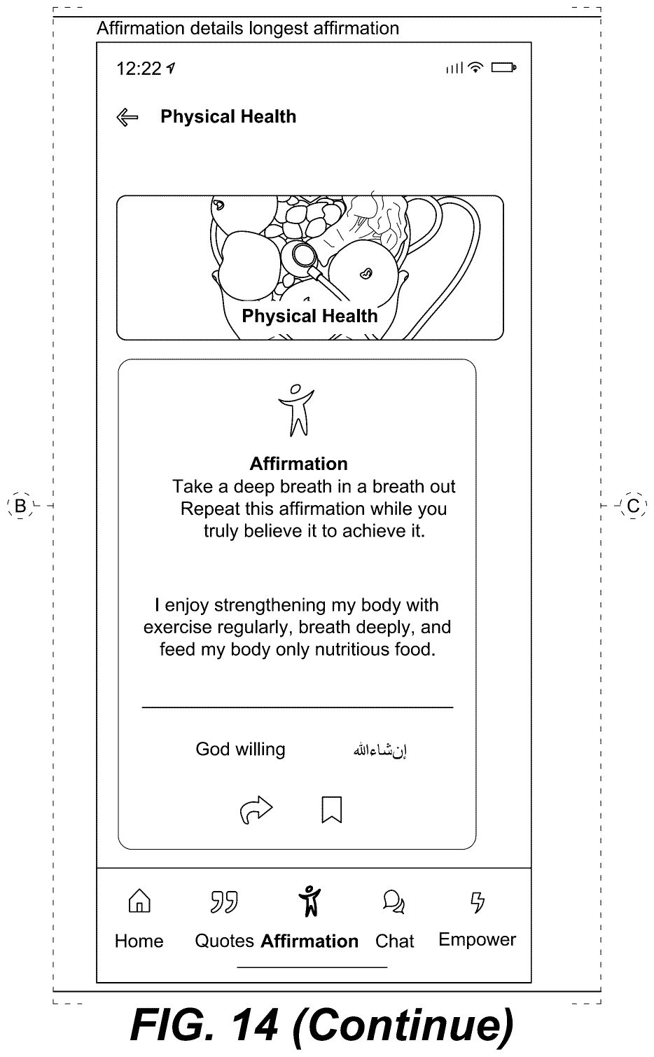

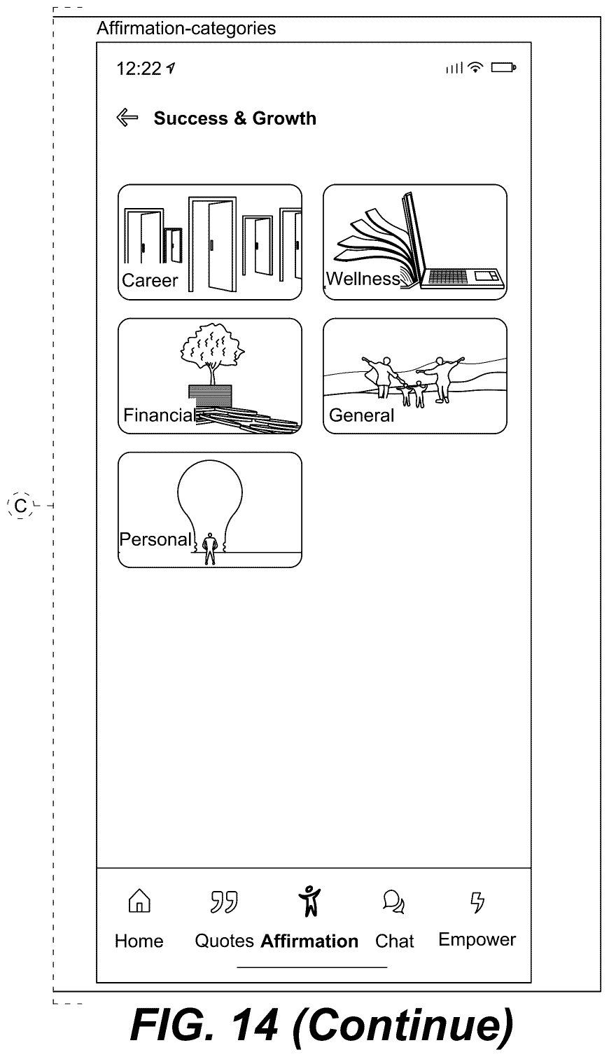

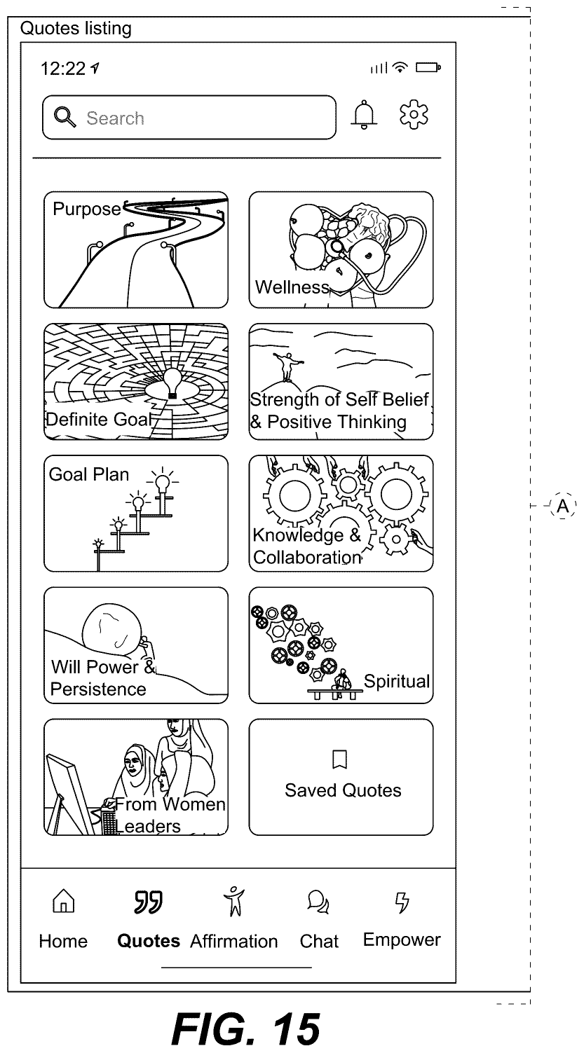

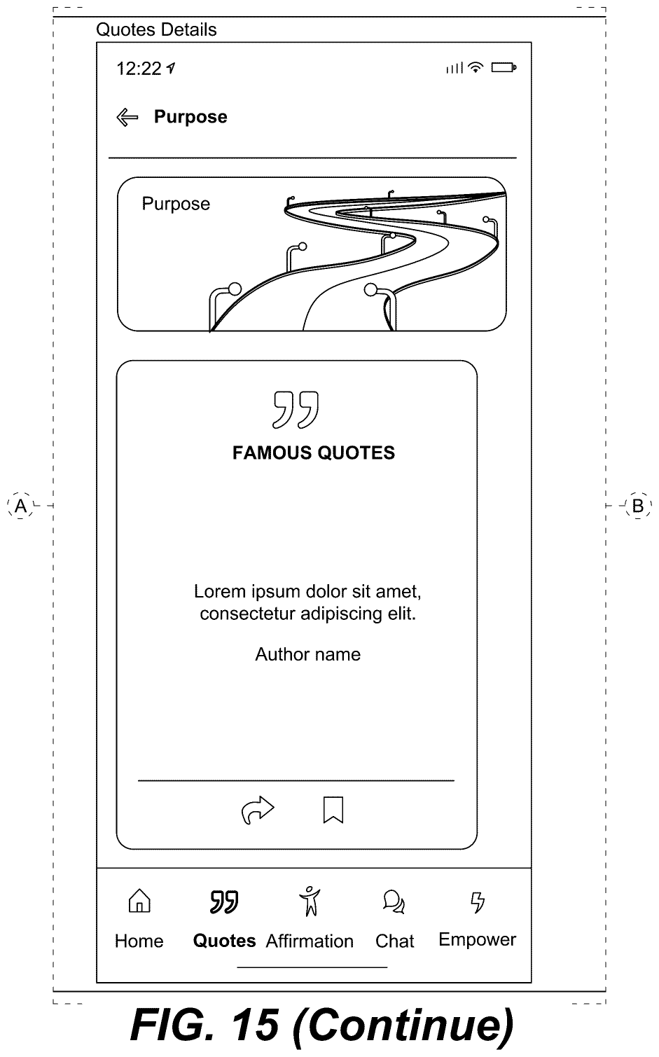

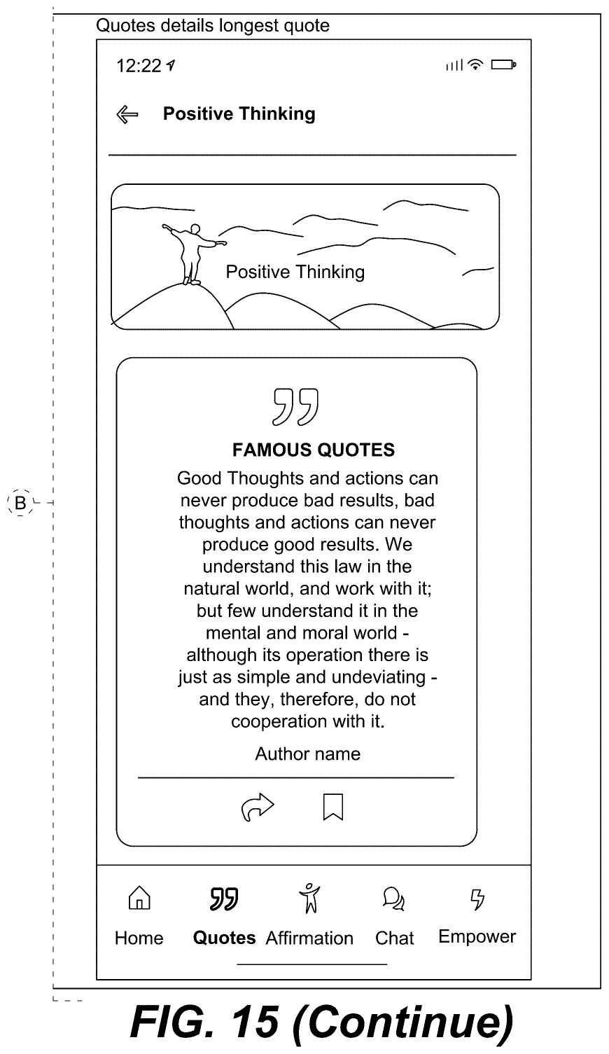

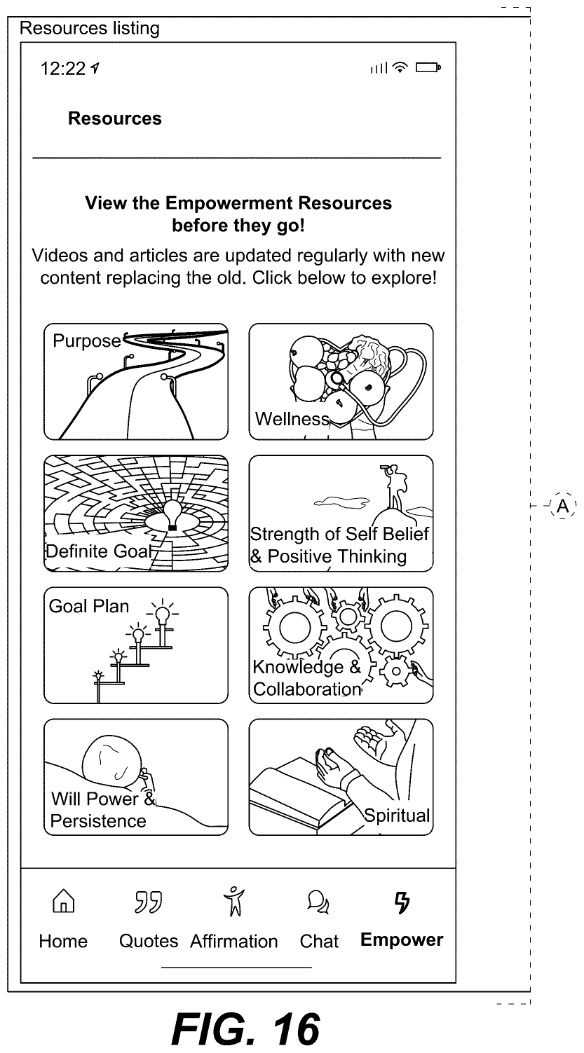

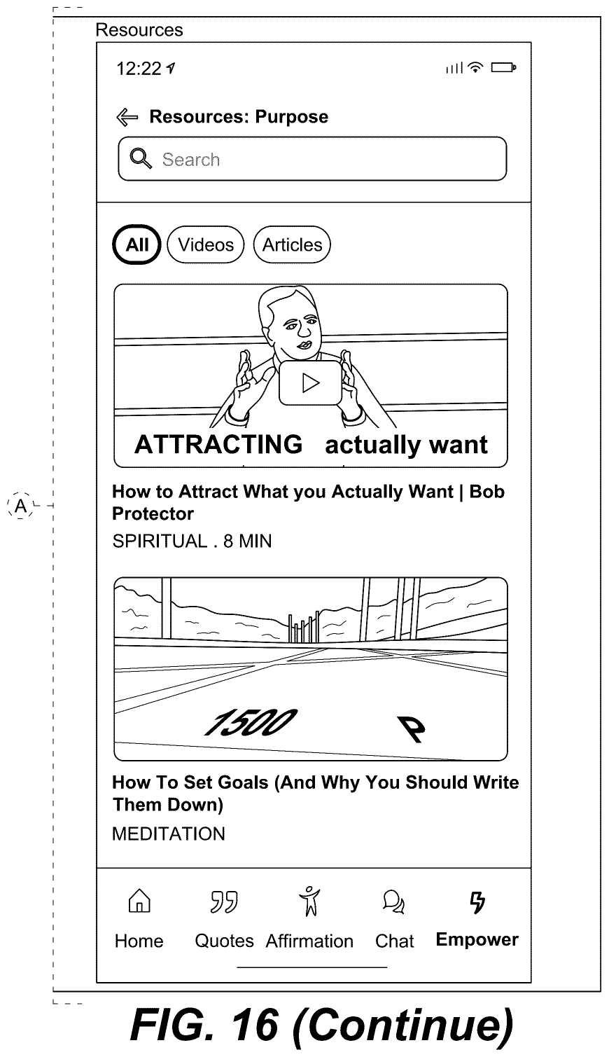

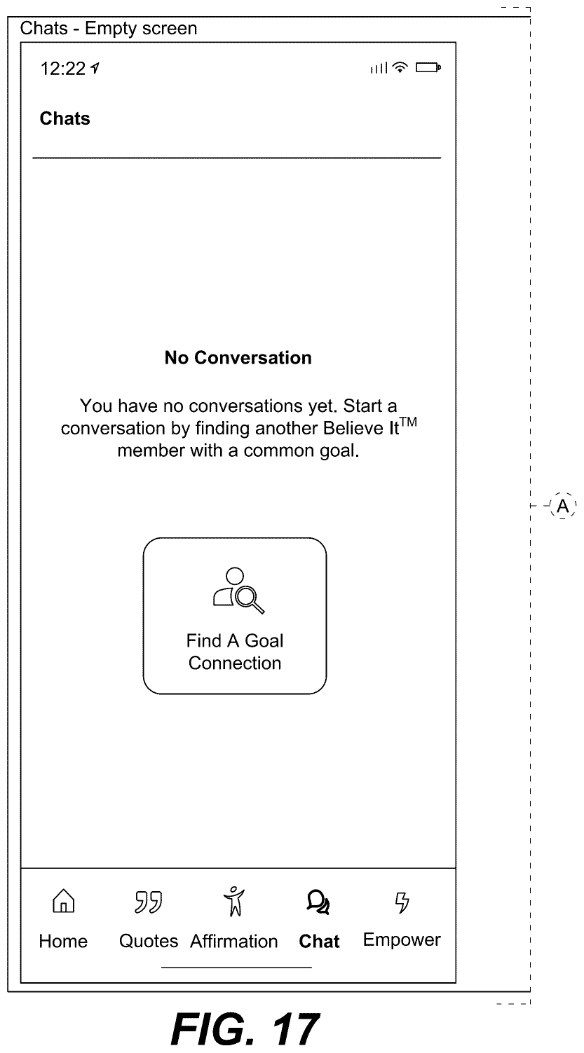

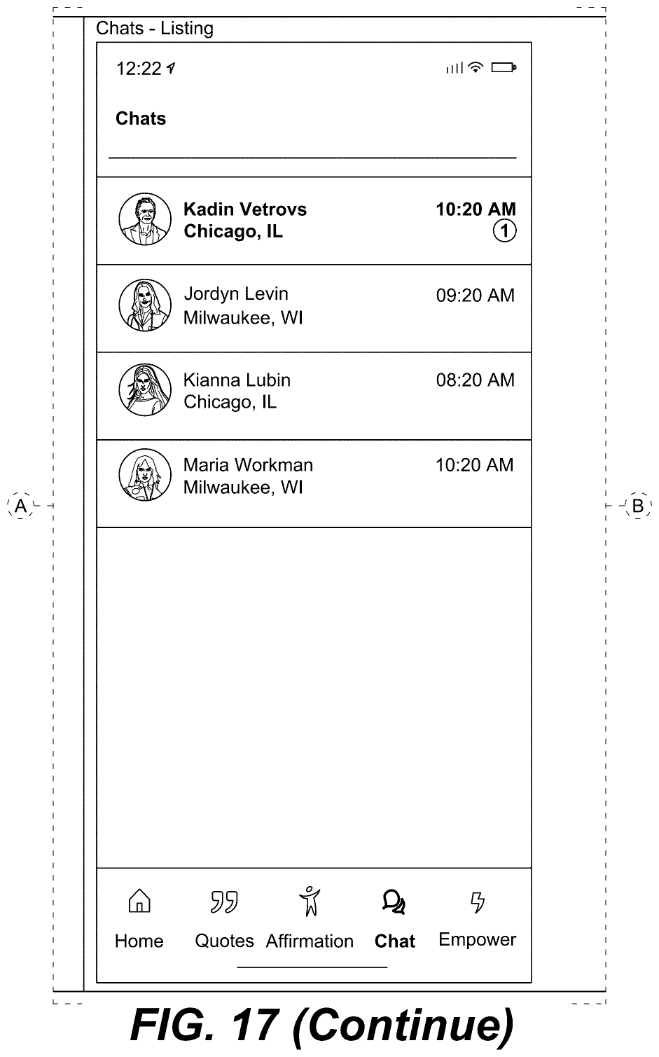

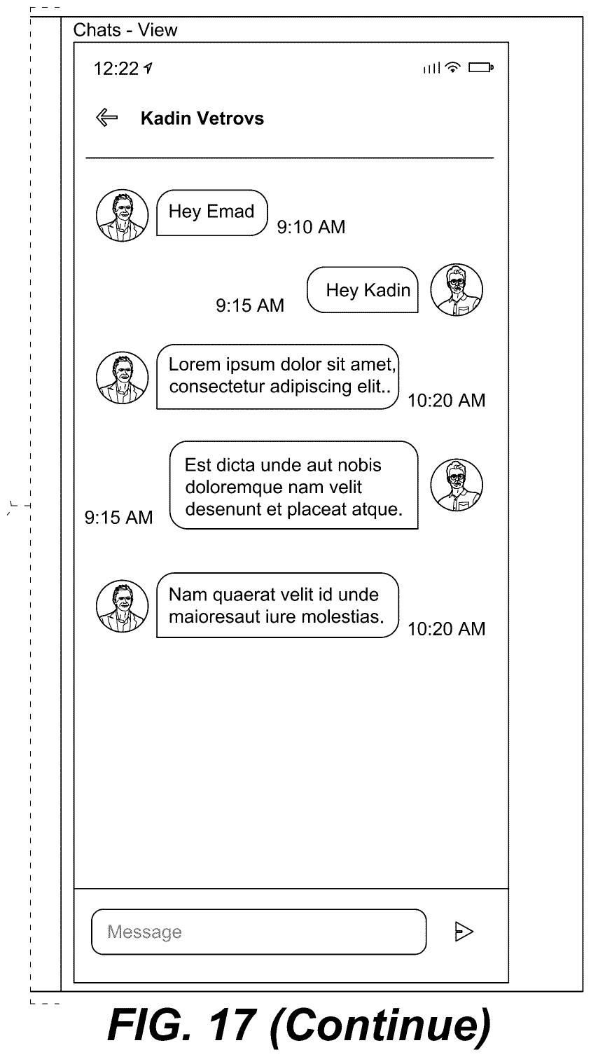

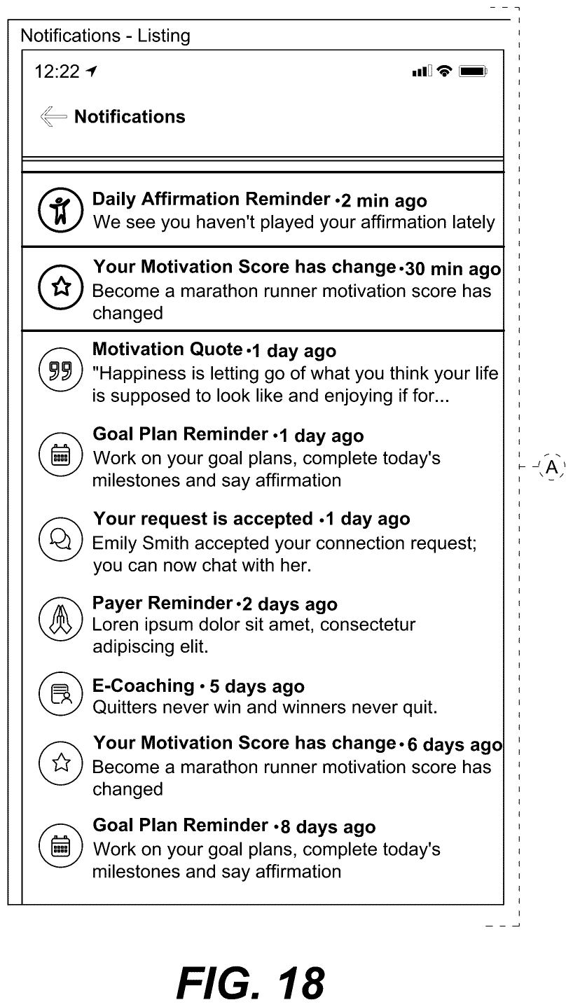

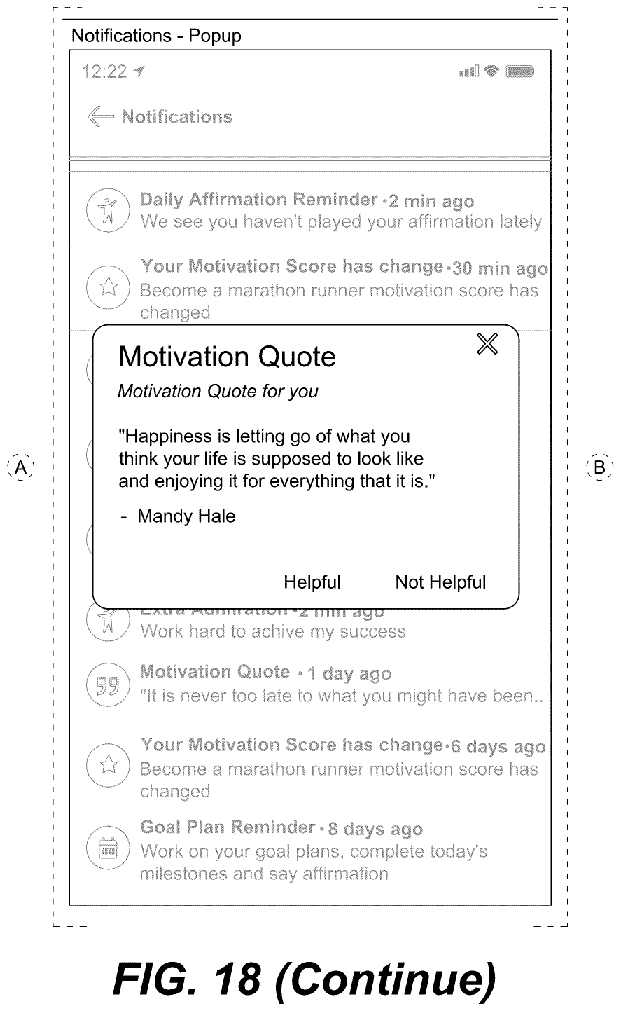

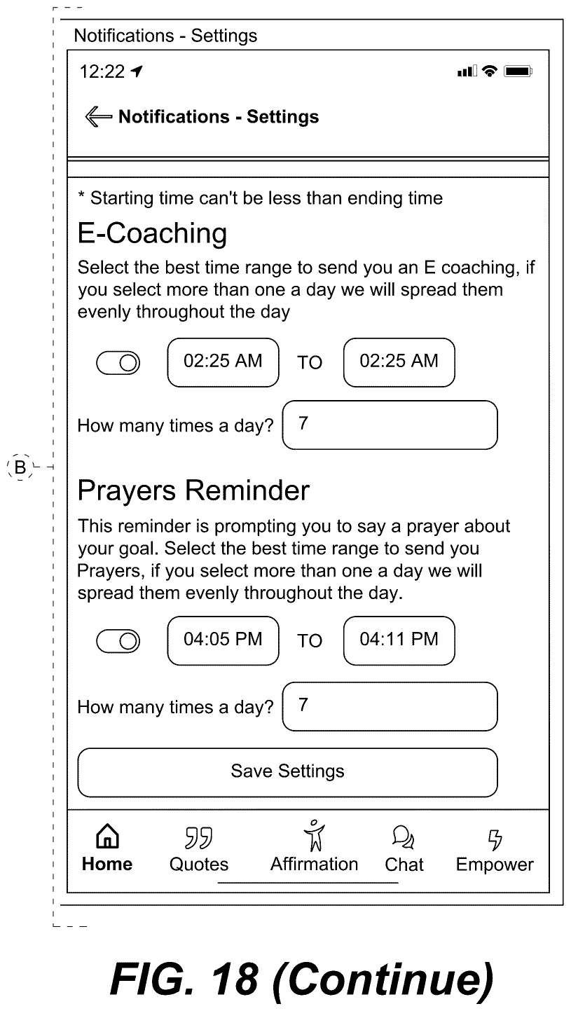

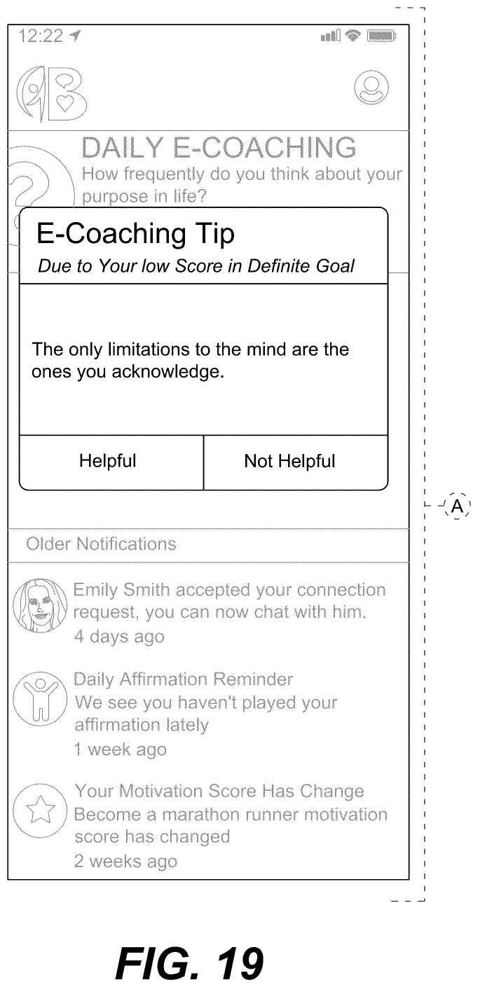

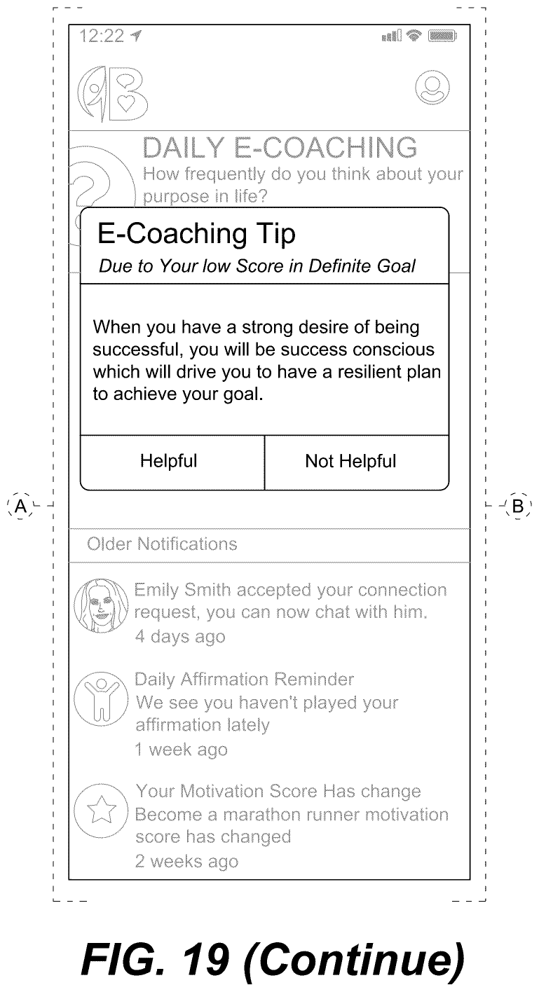

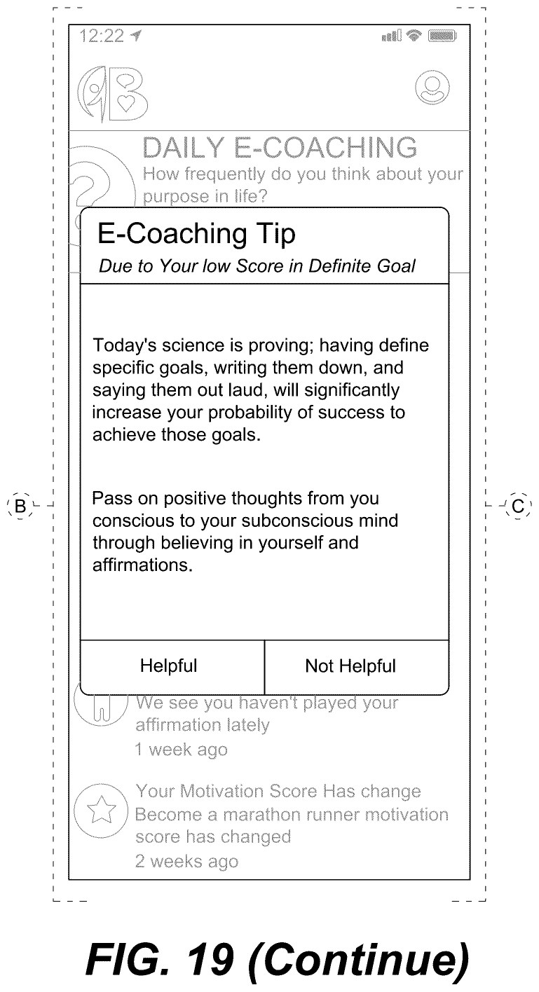

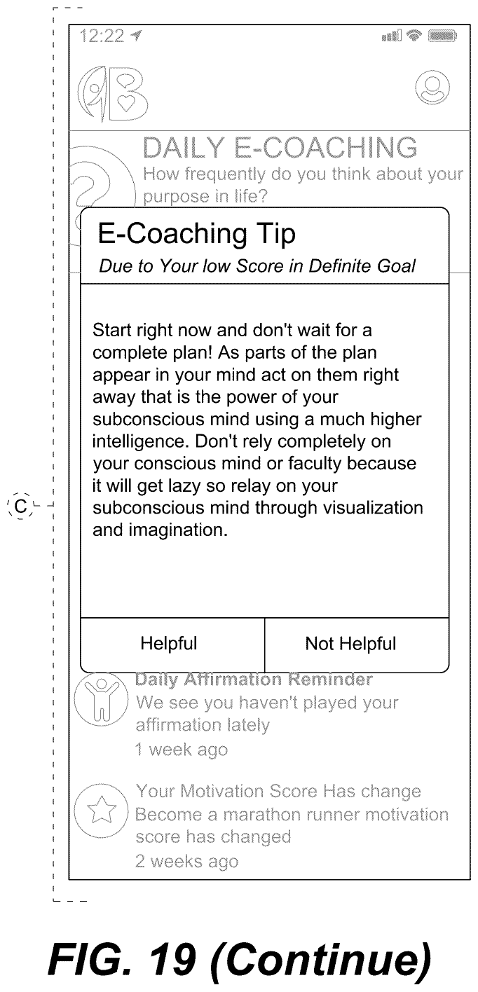

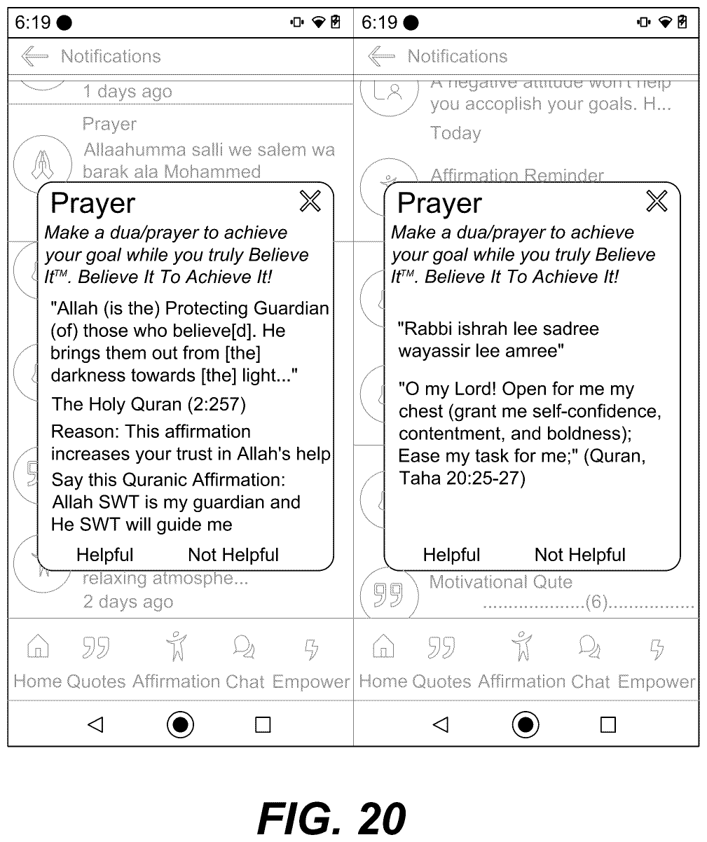

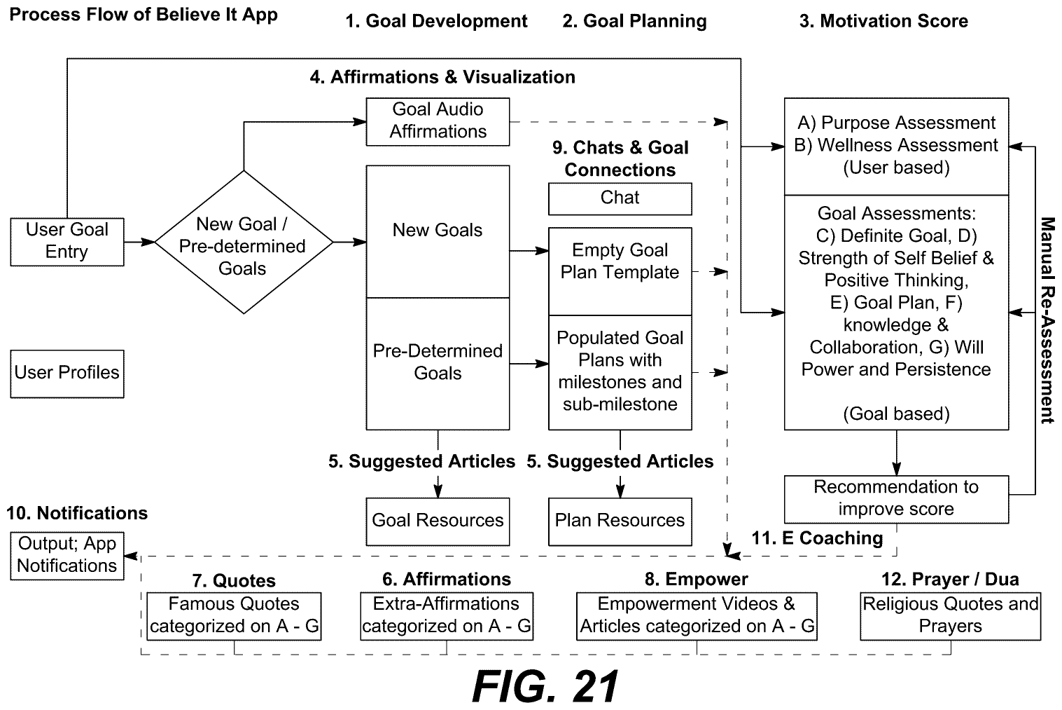

FIG. 1 illustrates an initial exemplary graphical user interface (GUI) for use with a mobile application for use with a mobile computing device, like a smartphone, a tablet, a professional digital assistant or other similar like device. Such an initial GUI may comprise a goal screen for use with a professional mobile application.

As illustrated, the GUI may require input from the user and may request information from a user by way of a plurality of query fields, for example, the user may be called upon to enter a goal name such as “Getting promoted” and may also be required to “Select goal category.” As just one example, the goal development helps the user to develop the definite goals properly with a number of query fields. Such query fields may also comprise one or more of the following fields: Goal name in the present tense format; i.e.; ‘I am a graduate student in top university’. The user may also be required to enter a date of accomplishment. In one preferred arrangement, such a date of accomplishment may have a temporal limitation, such as being at least 21 days from the entry date as majority of goals are difficult to achieve in less than 21 days.

The initial GUI may also require the user to enter an affirmation statement (i.e.; I am achieving my goal by certain date, and once I accomplish my goal, I will give back by helping others). The GUI may also request that the user respond to the following inquiry “Why is this goal important to you?” These queries serve multiple purposes, such as that they will help the user understand the intrinsic motivators of why they want to pursue and accomplish this specific goal.

FIG. 2 illustrates yet another exemplary image selection graphical user interface (GUI) for use with a mobile application for use with a mobile computing device, like a smartphone, a tablet, a professional digital assistant or other similar like device. This GUI may request that the user select a goal picture or some type of image that can be used to inspire them about the goal. As noted in FIG. 2, there might be two options for the user to select such a picture for inspiration. The GUI may provide the option for the user to select a picture or an image from an existing photo gallery provided by the mobile application. Or the user may be able to provide a picture from the user's own compute device. As those of ordinary skill in the art will recognize, alternative methods of photo and/or image selection may be utilized as well.

In one preferred arrangement, such a photo and/or image selection criteria step or process would operate so as to remind the users of why they are pursuing such a goal. In addition, it can also act to motivate the user when they are not motivated. In one preferred mobile application arrangement, this picture and/or image will be used in the affirmation feature of the mobile application to perform various affirmation and visualization exercises properly while looking at the picture or image and then visualizing as if the user already accomplished such a targeted goal. In one preferred arrangement, this picture can be taken from the mobile camera, uploaded via the mobile pictures or pre-selected by the goal type.

FIG. 3 illustrates yet another exemplary record audio affirmation graphical user interface (GUI) for use with a mobile application for use with a mobile computing device, like a smartphone, a tablet, a professional digital assistant or other similar like device. As illustrated, in one preferred arrangement, the mobile application based in part on this GUI asks for the user to provide an audio affirmation. In one preferred arrangement of the present disclosure, this audio affirmation comprises a recording of the users saying their affirmation statement while they are truly believing in themselves.

In one arrangement, the GUI provides the user with the capability of uploading an affirmation audio (music file) from the mobile as a goal music theme. As just one example, such an affirmation audio may comprise a music file. However, those of ordinary skill in the art will recognize, alternative affirmation audio arrangements and/or file types may be utilized as well. The functionality of the recording audio affirmation and uploading the goal audio music theme gives the user the ability to play their recording affirmation and music in the background to inspire them and to be utilized in the affirmation and visualization exercises as described herein in detail.

In addition, the GUI illustrated in FIG. 4 will also provide the user with the option of allowing this affirmation to be made public. That is, this GUI enables the user of the mobile application to make their affirmation to other users utilizing the mobile app platform. This GUI also allows the user to select a suggestion feature wherein the mobile application will generate articles and third-party resources for user notification. For example, the GUI may provide a slider so that a user can select the goal to be public or kept private. This provides multiple options and opportunities. For example, it provides the user the ability to have his or her stated goals to be searchable on the mobile application platform. As just one example, it also provides the ability to have his or her stated goals to be searchable on the mobile application platform by other users, chat via a mobile application social platform with other users, see their goals, and leverage best practices. As those of ordinary skill in the art will recognize, alternative uses and/or functions may also be utilized by such a public availability option.

FIG. 5 illustrates yet another exemplary public affirmation graphical user interface (GUI) for use with a mobile application for use with a mobile computing device, like a smartphone, a tablet, a professional digital assistant or other similar like device. In addition, the GUI illustrated in FIG. 5 also provides an optional user slider that allows or enables the mobile application to show suggested articles. This feature gives the users the ability to have a plurality of third-party resources that align with the user's goals. As just one example, the mobile app may be configured to provide the top three suggested articles from an API.

FIG. 6 illustrates an exemplary category identification graphical user interface (GUI) for use with a mobile application for use with a mobile computing device, like a smartphone, a tablet, a professional digital assistant or other similar like device. In this illustrated GUI, the mobile application allows users to enter a pre-determined goal. In one preferred arrangement, the mobile application provides a plurality of pre-determined goals. As just one example, in one arrangement, the mobile application provides about 60 “Pre-Determined Goals” and these goals may comprise goals of all types. As just one example, such goals may relate to one or more of the selectable icons illustrated in the GUI illustrated in FIG. 6. For example, these selectable icons may comprise or be related to certain goal-oriented topics such as career, financial, educational, academic, family goals and/or other types of goal-oriented items.

In one preferred arrangement, these pre-determined goals may be developed under the direction and supervision various types of professionals, like by certified coaches in these respective fields. In one preferred arrangement, each of these goals comprises one or more milestones along with one or more sub-milestones. These milestones are configured and structured for the user to follow in order to help them achieve their goals. As just one example, if the user selects any of these pre-determined goals, the goal plan features of the mobile application takes into an account certain item.

As just one example, the goal plan features may take one or more of the following into account: the accomplishment date, today's date and the goal milestones. Having taken these items into account, the mobile application will then show or illustrate a goal plan for the user to visualize and confirm. In addition, in one preferred arrangement, the mobile application will also provide and/or visually provide suggested dates for full and/or partial goal completion accordingly. If the user selects a pre-determined goal, in one preferred mobile app arrangement, the mobile app will automatically auto-populate the goal development features with one or more of the following items: the goal name, the goal category, and/or the goal picture or image.

FIG. 7 illustrates an exemplary goal planning graphical user interface (GUI) for use with a mobile application for use with a mobile computing device, like a smartphone, a tablet, a professional digital assistant or other similar like device. In one preferred arrangement, the mobile application provides for certain developed key milestones and sub milestones similar to those key milestones and sub-milestones illustrated in FIG. 7. The mobile application may also provide for certain developed key milestones and sub milestones with a daily planner and time tracking to achieve a user's goals. For example, in one preferred arrangement, the mobile application generates certain daily progress tracking features so that the user can monitor his or her progression while staying on track to achieve their stated goals. In this manner, the mobile app will help the user to nurture and foster the development of positive habits.

Progress tracking provided by the systems and methods disclosed herein can be important for several reasons. For example, the presently disclosed systems and methods will allow users to monitor and track your progress systematically to gain valuable insights into your journey of self-improvement. These systems and methods will allow users to analyze their achievements, identify areas for improvement, and celebrate milestones along the way.

In one preferred arrangement, the mobile application provides the user with access to more than 60+ predetermined goals, development by a certified career, educational, family and financial coaches. In addition, in one arrangement, the mobile application also provides features with plans to edit the dates and milestones based on a user's preference. Alternatively, the mobile application will allow users to customize their own goal plan. In addition, the mobile application will allow the user to access articles that help the user to develop and update his or her goal plan accordingly.

The mobile application also provides for a certain goal planner features for one or more pre-determined goals. The mobile application may take as input the user's selection of name of the goal and date of accomplishment, then it displays all the necessary milestones to accomplish such a goal while recommending a due date for each of the milestone. In one arrangement, where this might be called for based on the user selected goal and its input parameter, the mobile app will equally divide the number of days between the current date and date of anticipated accomplishment by the number of milestones. Then, the mobile app will then suggest and assign recommended date for each milestone.

For example, FIG. 8 illustrates an exemplary milestone graphical user interface (GUI) for use with a mobile application for use with a mobile computing device, like a smartphone, a tablet, a professional digital assistant or other similar like device. As noted in FIG. 8, the users have the ability to edit, modify, or change the name of the milestone, recommended date, and date of the week to work on it as well as to assign a goal connection or goal partner to each of the milestone. In addition, the GUI may also provide a “Add New Milestone” icon which will allow the user to enter a new milestone, thereby supplementing the milestones already viewable by the GUI illustrated in FIG. 8.

FIG. 8 illustrates an exemplary goal planning feature graphical user interface (GUI) for use with a mobile application for use with a mobile computing device, like a smartphone, a tablet, a professional digital assistant or other similar like device. For example, the presently disclosed mobile application also comprises a goal planning feature for user custom made goals. More specifically, this goal planning feature allows the user to develop the appropriate milestones and sub-milestones with specific dates to accomplish the overall goal. In one preferred arrangement, this feature may be utilized a project planning template to collect the user entries.

FIG. 9 also illustrates an exemplary suggested resource feature graphical user interface (GUI) for use with a mobile application for use with a mobile computing device, like a smartphone, a tablet, a professional digital assistant or other similar like device. In one preferred arrangement, the goal planner feature further comprises the feature or ability to show one or more suggested articles. In one arrangement, this goal planning feature gives the users the ability to have top three suggested articles from an API that searches and offers the user the top searches from certain preferred search engines. As just one example, these search engines, such as Bing or Google, using structured search queries. The mobile application will generate active URL links thereby enabling users to quickly and efficiently locate the suggested articles for further review.

FIG. 10 illustrates an exemplary motivation score graphical user interface (GUI) for use with a mobile application for use with a mobile computing device, like a smartphone, a tablet, a professional digital assistant or other similar like device. Importantly, the presently disclosed mobile app generates a user motivation score as illustrated in FIG. 10. For example, in one preferred arrangement, the mobile application provides the user with a real-time assessment of the overall motivation for specific goals. In one arrangement, the mobile application will generate an assessment that is developed by group of professionals, such as psychiatrics, psychologists, and coaches that work in unison to identify what the user needs to work on to achieve their goals. In addition, this group of professionals may also provide for practical recommendations so that the user can work to improve their goal objectives. In one arrangement, the mobile application starts to enable the user to take a self-assessment questionnaire on motivation to know what the user needs to improve upon and on what items or areas that they user should focus upon. In one preferred arrangement, the mobile application will be configured so as to allow the user to retake the assessment periodically, such as weekly or bi-weekly.

The mobile application will be configured to generate a motivation score based on user input. As just one example, the motivation score may be configured or structured so as to assess the user on a plurality of different inputs or categories. As just one example, the motivation score may be configured or structured so as to assess the user based on seven different categories to determine their overall motivation scores and these motivation scores will be evaluated in a plurality of different areas. As just one example, the motivation score will be evaluated in three areas: a purpose score (e.g., out of 100), a wellness score (e.g., out of 100), and a goal score (e.g., out of 500).

Once the user takes the assessment, the mobile application will generate a list of recommendations which is then viewed by the user. This list of recommendations will then be structured or laid out in order to improve the user's scores in the different category which will overall improve their overall motivation to pursue their goals. The assessment can be taken as frequently as possible. As just one example, the assessment may be repeated weekly or biweekly. In one preferred arrangement, one or more of the assessment questions will be pre-populated based on the user's activities on the mobile app using application cookies and may also implement AI/machine learning technology. There is considerable importance of these assessments from a number of perspectives, such as psychosocial and medical perspectives.

The wellness assessment of the motivation score helps evaluate one's potential capacity for change, and whether they have psychosocial constraints that may hinder their success towards achieving their desired goal. The main constraints the mobile application explores include the user's sleep, dietary habits, and exercise routine. Once baseline information is provided from the assessment, further guidance is provided on how one can further improve the above aspects in their life, and potentially enhance their chance in meeting their goals.

The goal assessment is related to the motivation to pursue a specific goal which relies on many factors such as but not limited to: definition, plan, positive thinking, knowledge, collaboration, will power and persistence. The purpose assessment is related to user's clarity on purpose which allows to develop the right goals which align to that purpose. Goal setting theory and task motivation have been evolving domains for many years. The presently disclosed mobile application highlights some of salient recommendations including goal specificity, action planning, timeframe, and goal evaluation. A defined goal directs attention and energy towards goal-relevant activities, which tends to improve persistence and away from potential distractions. Additionally, the factor of utilizing already obtained skills or adopting new ones through proper strategies can play an important role in goal achievement.

FIG. 11 illustrates an exemplary assessment question graphical user interface (GUI) for use with a mobile application for use with a mobile computing device, like a smartphone, a tablet, a professional digital assistant or other similar like device. Further exemplary questions and exemplary answer types that may be used in formulating a motivation score are provided below.

| | Wellness - (One Assessment Per | Answer | |
|---|---|---|---|
| | User) | Type | Answers Choices |
| | Sleep Habits: | | |
| 1 | Are you satisfied with your sleep? | User | Gauge; (1 No −5 Somewhat −10 |
| | | Answer | Yes) 1 = 0%, 5 = 50%, 10 = 100% |
| | Exercise Habits: | | |
| 2 | Over the last month, have you | User | Multiple Choices: |
| | participated in regular exercise? | Answer | Never, Often, Weekly, 2-3 times |
| | | | per week, Daily (0, 30, 60, 80, 100) |
| | Substance Use: | | |
| 3 | Over the last two weeks, which of the | User | Multiple Choices: |
| | following substances have you used? | Answer | None, Caffeine, Alcohol, Nicotine, |
| | (Substance Use) | | Cannabis, Illicit drugs (Check all |
| | | | that apply), Other: (none = 100, and |
| | | | adding each items take 20 points |
| | | | out) |
| | Misc: | | |
| 4 | Over the last month, how would you | User | Gauge; 0 −10 (0 = Bad, 5 = |
| | rate your overall mood? | Answer | Average, 10 = Great) |
| 5 | How tolerant are you towards change? | User | Gauge; (0 = not tolerant, 5 = |
| | | Answer | Indifferent, 10 = excited about it) |
| 6 | What are you grateful for? | User | Multiple choices; which of the |
| | | Answer | following are you grateful for |
| | | | (Health/Wellness/Fitness, |
| | | | Educational, Professional /Career, |
| | | | Financial, Spiritual, check all that |
| | | | apply) each item is worth 20 points |

| Q | Purpose - (One Assessment Per | Answer | |
|---|---|---|---|
| # | User) | Type | Answers Choices |
| 1 | Have you found your life purpose? Do | User | Gauge; (1 No −5 Somewhat −10 |
| | you understand it? | Answer | Yes) 1 = 0%, 5 = 50%, 10 = 100% |
| 2 | How frequently do you think and | User | Gauge; (1 Never −5 Somewhat −10 |
| | reflect on your life purpose? | Answer | Always) 1 = 0%, 5 = 50%, 10 = |
| | | | 100% |
| 4 | Do your goals align with your life | User | Gauge; (1 No −5 Somewhat −10 |
| | purpose? | Answer | Yes) 1 = 0%, 5 = 50%, 10 = 100% |
| 5 | Are you often “stuck in the past” or | | Gauge; (1 Past or Future, 10 = |
| | are you constantly thinking about the | | Present) 1 = 0%, 5 = 50%, 10 = |
| | future? Past-oriented or future- | | 100% |
| | oriented? | | |

| Q | Definite Goal - (One Assessment | Answer | |
|---|---|---|---|
| # | Per Goal) | Type | Answers Choices |
| 1 | How important is your goal to you? | User | Gauge; (1 Yes −5 Somewhat −10 |
| | | Answer | No) 1 = 0%, 5 = 50%, 10 = 100% |
| 2 | How committed are you to this goal? | User | Gauge; (1 No −5 Somewhat −10 |
| | | Answer | Yes) 1 = 0%, 5 = 50%, 10 = 100% |
| 3 | How often do you think about your | User | Gauge; (1 No −5 Somewhat −10 |
| | goal? Do you think about it in the | Answer | Yes) 1 = 0%, 5 = 50%, 10 = 100% |
| | morning and evening? If so, how | | |
| | much? | | |
| 4 | Do you have a clear idea on how to | User | Gauge; (1 No −5 Somewhat −10 |
| | achieve your goal? | Answer | Yes) 1 = 0%, 5 = 50%, 10 = 100% |
| 5 | Are you dedicating enough time to | User | Gauge; (1 Yes −5 Somewhat −10 |
| | achieve it? | Answer | No) 1 = 0%, 5 = 50%, 10 = 100% |
| Q | Strength of Self Belief & Positive | Answer | |
| # | Thinking - (Goal) | Type | Answers Choices |
| 1 | If the user plays the affirmations | Via App | 0--14 = 0% −−> 100. 1 affirmation = |
| | 14/day | | (100/14) |
| | How many times did you say your | User | Gauge; Choices are 0-14 = 0% −−> |
| | affirmations aloud today? (this | Answer | 100. 1 affirmation = (100/14) |
| | question to be shown to the user) | | |
| 2 | How often do you concentrate on your | User | Gauge; Choices are 0−14 = 0% −−> |
| | thoughts and visualize that you have | Answer | 100. 1 affirmation = (100/14) |
| | already achieved your goals and | | |
| | become the person you want to be? | | |
| | Close your eyes and visualize | | |
| | yourself, while repeating your | | |
| | affirmations. | | |
| 3 | Do you believe you will achieve this | User | Gauge; (1 Not Confident −5 |
| | goal? | Answer | Somewhat −10 Very Confident) 1 = |
| | | | 0%, 5 = 50%, 10 = 100% |
| 4 | Do you think about your past | User | Gauge; (1 = Never, 5 = Somewhat |
| | successes? | Answer | frequently, 10 = Frequently); 1 = |
| | | | 0%, 5 = 50%, 10 = 100% |
| 5 | How badly has recent criticism about | User | Gauge; (1-impacted me very badly |
| | your goal affected you? | Answer | 5-somewhat impacted me 10- |
| | | | Didnt't impact me at all because |
| | | | they are just noise to me); 1 = 0%, |
| | | | 3 = 50%, 5 = 100% |
| 6 | Do you think positively about your | User | (1 = Never, 5 = Somewhat |
| | goal? | Answer | frequently, 10 = Frequently) |
| | | | 1 = 0%, 5 = 50%, 10 = 100% |
| 7 | Do you think negatively about your | User | (1 = Frequently, 5 = Somewhat |
| | goal? Do you have fears holding you | Answer | frequently, 10 = Never) |
| | back from moving toward this goal?” | | 1 = 0%, 5 = 50%, 10 = 100% |
| Q | | Answer | |
| # | Goal Plan - (Goal) | Type | Answers Choices |
| 1 | Have you developed a solid plan for | User | Gauge; (1-100) |
| | your goal? | Answer | |
| 2 | What percent of the action items on | User | Gauge; (1-100) |
| | your goal plan have specific start and | Answer | |
| | end dates? (this question to be shown | | |
| | to the user) | | |
| 3 | What percent of the action items on | User | Gauge; (100-0%) |
| | your goal plan are past deadline? | Answer | |
| 4 | Do you need to continue building and | User | Gauge; (1 No −5 Somewhat −10 |
| | improving your goal plan? | Answer | Yes) 1 = 0%, 5 = 50%, 10 = 100% |
| 5 | Do you analyze your goal plan (daily, | User | Gauge; (Never = 0, Monthly = 50, |
| | weekly, monthly or yearly) and take | Answer | Weekly = 75, Daily = 100) |
| | the necessary corrective actions | | |
| | regularly? | | |
| 6 | Are you looking for shortcuts to | User | Gauge; (1 Yes −5 Somewhat −10 |
| | success or are you diligently | Answer | No) 1 = 0%, 5 = 50%, 10 = 100% |
| | following your plan? | | |
| Q | Knowledge & Collaboration - | Answer | |
| # | (Goal) | Type | Answers Choices |
| 1 | Are you getting the necessary | User | Gauge; (1 No −5 Somewhat −10 |
| | knowledge to achieve your goal? | Answer | Yes) 1 = 0%, 5 = 50%, 10 = 100% |
| 2 | What percent of the action items on | User | Gauge; (1-100) |
| | your goal plan have an owner either | Answer | |
| | yourself or someone your are | | |
| | collaborating with? | | |
| 3 | If you have a team or are working | User | Gauge; (1 No −5 Somewhat −10 |
| | with someone else on your goal, are | Answer | Yes) 1 = 0%, 5 = 50%, 10 = 100% |
| | you providing them with the | | Or Working Along, then this score |
| | necessary directions to progress on | | is automatically a 100. |
| | this goal? | | |
| 4 | If you have a team or are working | User | Gauge; (A Not interact, B Monthly, |
| | with someone else on your goal, how | Answer | C Weekly, D Couple times per |
| | frequently do interact? | | week) |
| | | | (A = 0%, B = 33%, C = 67%, D = |
| | | | 100%) |
| | | | Or Working Along, then this score |
| | | | is automatically a 100. |
| 5 | If you have a team or working with | User | Gauge; (1 No −5 Somewhat −10 |
| | someone on your goal, do you all | Answer | Yes) 1 = 0%, 5 = 50%, 10 = 100% |
| | have the same values and work in | | |
| | harmony? | | |
| 6 | If you have a team or are working | User | Gauge; (1 No −5 Somewhat −10 |
| | with someone else on your goal, is | Answer | Yes) 1 = 0%, 5 = 50%, 10 = 100% |
| | your plan approved by them ? | | |
| 7 | How decisive is everyone working on | User | Gauge; (1 = Procrastinating, 3 = Not |
| | the plan? | Answer | quick enough, 10 = Fairly quickly) |
| | | | 1 = 0%, 5 = 50%, 10 = 100% |
| Q | | Answer | |
| # | Will Power and Persistence - (Goal) | Type | Answers Choices |
| 1 | If the user plays the affirmations | Via App | 0--14 = 0% −−> 100. 1 affirmation = |
| | 14/day | | (100/14) |
| 2 | How many times did you say your | User | Gauge; Choices are 0−14 = 0% −−> |
| | affirmations aloud today? | Answer | 100. 1 affirmation = (100/14) |
| | How often do you concentrate on your | User | Gauge; Choices are 0--14 = 0% −−> |
| | thoughts and visualize that you have | Answer | 100. 1 affirmation = (100/14) |
| | already achieved your goals and | | |
| | become the person you want to be? | | |
| | Close your eyes and visualize | | |
| | yourself, while repeating your | | |
| | affirmations. | | |
| 3 | What percent of the action items on | User | Gauge; (100-0%) |
| | your goal plan are past deadline? (this | Answer | |
| | question to be shown to the user) | | |
| 4 | Do you need to continue building and | User | Gauge; (1 No −5 Somewhat −10 |
| | improving your goal plan? (this | Answer | Yes) 1 = 0%, 5 = 50%, 10 = 100% |
| | question to be shown to the user) | | |
| 5 | Do you acknowledge your mistakes or | User | Gauge; (A- Tend to blame others, |
| | blame others? | Answer | B- In−Between, C - Accept failures) |
| | | | A = 0%, B = 50%, C = 100 |

The present mobile application contains numerous affirmations and visualizations and provided user access to hundreds of positive affirmations, with Insha'Allah/God willing included. These affirmations are made available in different categories for daily motivation and increase of a user's self-belief and self-confidence to achieve their goals. Develop positive thinking through habit formation. The use of such affirmations and visualizations is well documented. For example, there are many public articles that explains the psychological aspects and benefits. For example, “They encourage you to visualize and believe a statement to make positive changes in your life. Affirmations are phrases that you repeat to yourself to change your subconscious thoughts. Over time, they replace any negative beliefs or thoughts with positive thoughts. This instils confidence, positivity, and ambition.”

The presently disclosed mobile app comprises a feature wherein this feature empowers the user to override their subconscious mind by visualizing themselves already achieving their goal. This allows users to repeat their own affirmation statement that they wrote in the goal development feature which was provided and discussed herein with respect to FIG. 1. (i.e.; I am achieving my goal by certain date, and once I accomplish my goal, I will give back by helping others).

For example in FIG. 12, in one arrangement, the mobile application provides the user with certain steps that instructs the user as to how they can use their daily affirmation. As just one example, the mobile application provides the following suggested steps. First, find a quiet place or a place to motivate you to say your affirmations aloud. Then, relax your mind and body. Thereafter, repeat your statement from the application “I Am . . . ” by “Accomplished By Date” God willing and “After I accomplish My Goal . . . . I will give back by . . . ”. Then, repeat your affirmations aloud for at least 7 times when you just wake up very early in the morning, and at least 7 times right before you go to sleep or for 10 minutes morning and night. Then, close your eyes and imagine yourself as if you already achieved your goal. As if you are seeing your goal, touching it, and living it. Invest all of your emotions and senses as you say your affirmations. Without emotions and feelings, the affirmations will not have any effects on you or your actions. This will help you override your subconscious mind with positive thoughts about your goal. You must also believe in yourself. Have a mindset of an absolute belief (not a maybe mindset) that you already achieved and will achieve your goal. As new parts of your plan appear in your mind during the affirmations, you need add them to your goal plan and act on them right way. For additional effectiveness, handwrite your affirmations down as you say them for the first time.

In addition, in one GUI arrangement as illustrated in FIG. 12, the mobile application will ask the user to play their recorded affirmations repeatedly. For example, in this GUI implementation, the mobile app will ask the user to repeat them at the same time while looking at the goal picture for at least 7 times in early morning and 7 times late in evening. As illustrated, the mobile application provides options to play and repeat their affirmations. For example, in the illustrated GUI example, these options include play recorded affirmations only, play audio music file only, play both recorded affirmations and audio file. However, as those of ordinary skill in the art will recognize, alternative affirmation playback may also be utilized.

The potential importance of affirmations (and visualization if possible) cannot be overstated, especially from a medical perspective. For example, self-affirmations are acts often involving the assertion of one's core values, by which protecting one's sense of self against threats against their integrity or competence. Affirmations can have positive effects on reducing stress, becoming more amendable for behavioral change, and improving academic performance. Hence, the presently disclosed mobile application attempts to harness this vehicle towards improving one's chances of achieving their goals.

FIG. 13 illustrates an exemplary goal resources graphical user interface (GUI) for use with a mobile application for use with a mobile computing device, like a smartphone, a tablet, a professional digital assistant or other similar like device. In one preferred arrangement, the disclosed mobile application generates a list of suggested articles based in part on the user's selected goal or goals. For example, as illustrated in FIG. 13, the mobile app provides users with the top articles from public domains that may be related to the user's goal.

In one arrangement, the mobile app will perform a search based on the previously noted goal development feature and search queries may be generated. For example, in one arrangement, the mobile app may generate a search based on search queries. Based on these search results, the user can read and forward such documents to other users and anyone on the user's contact list.

As illustrated in FIG. 14, the mobile application will also generate positive affirmations for users to use. It will also provide access to hundreds of positive affirmations, including Islamic words such as Insha'Allah/God-willing, in different categories to increase the user self-belief and self-confidence in achieving the user's goals. One of the primary purposes for providing such positive extra affirmations to the user is to enable the user to develop positive thinking through habit formation with daily affirmations.

Referring back to one preferred mobile application arrangement, content can be updated via the mobile app's administrative panel. In one arrangement, the content is publicly available but has been vetted out to improve the user overall motivation. In one preferred arrangement, these affirmation categories include confidence, decision making, fitness and weight loss, gratitude, happiness, kids empowerment and motivation, letting go, mental health and strength, mindfulness, overcome negativity and hard times, physical health, prosperity, relationships, self-esteem, self-love, success and growth, women empowerment, work and productivity, and daily life.

Referring back to FIG. 14, the mobile application allows the application user to view various affirmation statements. In one arrangement, the mobile application allows the user with the ability to slide right or left while saving their favorite affirmation statement. In addition, in one arrangement, the user can read and forward the affirmations to other users and anyone on their contact list. The GUI also provides the user with functionality instructing the user to take a deep breath in and breath out. Repeat this affirmation statement while you truly believe it. Say “God Willing” in English or “Insha'Allah” in Arabic.

FIG. 15 illustrates an exemplary famous items graphical user interface (GUI) for use with a mobile application for use with a mobile computing device, like a smartphone, a tablet, a professional digital assistant or other similar like device. In one arrangement, the mobile application will also provide the user with access to hundreds of motivational quotes and inspirational quotes from different successful leaders to inspire you throughout your day. And these thoughts might be categorized in multiple user selectable libraries. Such libraries may be categorized in accordance with subjects such as wellness, purpose, definite goals, strength of self-belief and positive thinking, goal plan, knowledge and collaboration, willpower and persistence, from women leaders, and spiritual, general motivational quotes. In one arrangement, the content is publicly available but has been vetted out to improve the user overall motivation in the same motivate score categories. As just one example, the mobile app GUI will provide access to Quran verses to empower and motivate the user while being mindful of God. In one arrangement, this content can be updated via the app administrative panel. The user has the ability to slide right or left while saving their favorite affirmation statement. In one arrangement, the GUI allows the user can read and forward the quotes to other users and anyone on their contact list.

FIG. 16 illustrates an exemplary Empower feature graphical user interface (GUI) for use with a mobile application for use with a mobile computing device, like a smartphone, a tablet, a professional digital assistant or other similar like device. For example, the mobile application provides the user access to a library of empowering articles and videos on self-help, Islamic self-improvement, and productivity. In one preferred arrangement, the library comprises content that has been regularly updated frequently to inspire and motivate the user to achieve their goals. In one preferred arrangement, library content can be updated via the app administrative panel. In one preferred arrangement, the library content is publicly available but has been vetted out to improve the user overall motivation in the same motivate score categories. As just on example, these motivation score categories may comprise one or more of the following categories: wellness, purpose, definite goal, strength of self-belief and positive thinking, goal plan, knowledge and collaboration, willpower and persistence. In one preferred arrangement, the mobile application allows users to read and forward the quotes to other users and anyone on their contact list. Other articles and videos are made by the Believe It team to educate the user on how to improve their overall motivation scores specifically in these categories: purpose, wellness, definite goal, strengthening of self believe and positive thinking, goal plan, knowledge and collaboration, and will power and persistence.

With the Empower Feature Library, users will be provided access a comprehensive resource library curated by certified coaches and experts. This will enable users the ability to explore a wide range of educational materials, practical tools, and insightful content tailored to your specific goals and needs. Users will benefit from the integration of Islamic values in the resources, enabling users to align their personal growth with their faith.

FIG. 17 illustrates an exemplary chat platform graphical user interface (GUI) for use with a mobile application for use with a mobile computing device, like a smartphone, a tablet, a professional digital assistant or other similar like device. As illustrated, in one preferred GUI arrangement, the mobile application is configured to provide a chat platform and goal connections platform. With such a feature, the mobile app allows users to connect with other users who share similar goals and exchange ideas. This will allow users to leverage best practices and create a support community to help each other achieve success. As just one example, the mobile app GUI interface can be configured so as to allow a user to search for and identify other mobile application users under each development goal, the mobile application has the ability to search users within the area or globally by their names or proximity. As just one example, the user may utilize the GUI as illustrated is to send a friend request once a contact is found. Once the friend request has been accepted, the user can utilize the GUI to chat with the new connection and/or friend and then add that new friend to a specific goal plan milestone.

Users will therefore connect with a supportive community of like-minded users who share similar goals and aspirations. Users will thereby engage in discussions, exchange ideas, and provide mutual support to help each other achieve success.

FIG. 18 illustrates an exemplary notifications graphical user interface (GUI) for use with a mobile application for use with a mobile computing device, like a smartphone, a tablet, a professional digital assistant or other similar like device. This notifications GUI allows users to customize the notifications to receive E-Coaching and build success habits, with either one, three, five, or seven notifications per feature. For example, this GUI can be configured to allow a user to set the number of notifications that suit the user needs across all app features to ensure staying on track towards achieving their goals. This notification feature essentially customizes the app journey for the user, i.e. coaching on demand to be coached on your goal as frequently as the user needs.

These notifications and reminders will allow users to stay on top of their goals with personalized notifications and reminders. Users can receive timely prompts and encouragement to take action and stay focused on their aspirations.

FIG. 19 illustrates an exemplary E-Coaching graphical user interface (GUI) for use with a mobile application for use with a mobile computing device, like a smartphone, a tablet, a professional digital assistant or other similar like device. This exemplary notification GUI also comprises E-Coaching Statements in a form of push notifications. For example, the mobile app can serve as a user's accountability coach, giving the user the coaching recommendations throughout the day based on their goal motivation score until they achieve their goal and move on to the next. For example, in one exemplary mobile app arrangement, the mobile app may comprise a database on E-Coaching statements and recommendations in the backend of the mobile app.

In one arrangement, the E-Coaching statements may be sent out to the user based on their specific motivation score in the different categories, i.e. the user will get recommendations on wellness if they score low in the wellness category of the motivation score. On the other hand, if a user has a perfect score in the purpose category, they will not receive any E-Coaching statements and recommendations. For the E-Coaching recommendations to be accomplished, then the user should re-take the assessment test to get their new motivation score which will generate a different set of eE-Coaching recommendations based on the new score.

FIG. 20 illustrates an exemplary prayers graphical user interface (GUI) for use with a mobile application for use with a mobile computing device, like a smartphone, a tablet, a professional digital assistant or other similar like device. For example, this GUI is configured to prompt the user to say a prayer about their goal. As just one example, the mobile app may comprise a database of an Islamic inspirational and motivational Quranic verses, prophetic hadiths, and prayers to be send to the user based on their selected notification time duration.

FIG. 21 illustrates an exemplary process flow diagram for use with a mobile application for use with a mobile computing device, like a smartphone, a tablet, a professional digital assistant or other similar like device.

By offering these comprehensive features, the presently disclosed systems and method empowers individuals to embrace a holistic approach to self-improvement while aligning their efforts with Islamic principles. Through goal setting, motivation assessment, positive affirmations, community support, E-Coaching, and a rich resource library, the disclosed systems and methods provide a robust platform for users to achieve their goals and lead a fulfilling life of growth and success.

The description of the different advantageous embodiments has been presented for purposes of illustration and description and is not intended to be exhaustive or limited to the embodiments in the form disclosed. Modifications and variations will be apparent to those of ordinary skill in the art. Further, different advantageous embodiments may provide different advantages as compared to other advantageous embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

We claim:

1. A method for empowering a user, comprising the steps of:
establishing a plurality of pre-determined goals;
storing a plurality of milestones related to each of the plurality of predetermined goals;
having a user announce a stated goal;
determining whether the stated goal comprises one of the plurality of pre-determined goals;
if the stated goal comprises one of the plurality of pre-determined goals, using the previously stored milestones;
if the stated goal does not comprise one of the plurality of pre-determined goals, having the user establish a customized goal;
if establishing customized goal, the method comprises the steps of
developing a plurality of customized milestones and
developing a plurality of customized sub-milestones;
the method further comprises the steps of
having the user state a date of accomplishment; and
having the user state an affirmation statement.

2. The method of claim 1 further comprising the step of having the user state an importance of the stated goal.

3. The method of claim 1 further comprising the step of generating a reminder image that operates to remind the user of the stated goal.

4. The method of claim 1 further comprising the step of performing an audio affirmation of recording the users stating an affirmation statement.

5. The method of claim 1 further comprising the step of uploading an affirmation audio file.

6. The method of claim 1 further comprising the step of selecting whether the stated goal will comprise a public stated goal or a private stated goal.

7. The method of claim 6 further comprising the step of allowing the stated goal and the user to be searchable.

8. The method of claim 1 further comprising the step of generating a motivation score.

9. The method of claim 8 wherein the motivation score comprises a purpose score, a wellness score, and a goal score.

10. The method of claim 8 wherein the motivation score is based in part on a user self-assessment questionnaire.

11. The method of claim 8 further comprising the step of providing a recommendation list to the user based in part on the motivation score.

12. The method of claim 8 further comprising the step of providing a plurality of sorted publicly available videos and articles based in part on the motivation score.

13. The method of claim 1 further comprising the step of providing an E-Coaching notification based in part on the motivation score.

14. The method of claim 11 wherein the recommendation list comprises a plurality of recommended articles.

15. The method of claim 1 further comprising the step of performing an affirmation exercise.

16. The method of claim 1 further comprising the step of performing visualization exercises.

17. The method of claim 1 further comprising the step of suggesting articles for assisting the user to achieve the stated goal.

18. The method of claim 1 further comprising the step of assisting the user with learning about developing a plan and achieving the stated goal.

19. The method of claim 1 further comprising the step of providing a plurality of readable affirmation.

20. The method of claim 1 further comprising the step of providing access to affirmations and visualizations.

21. The method of claim 1 further comprising the step of repeatedly playing the user's affirmations.

22. The method of claim 1 further comprising the step of providing extra affirmations.

23. The method of claim 1 further comprising the step of providing a plurality of motivational tools.

24. The method of claim 23 wherein the motivational tools are selected from the group comprising motivational quotes and inspirational quotes.

25. The method of claim 1 further comprising the step of providing a social platform for exchanging ideas.

26. The method of claim 1 further comprising the step of establishing a chat platform for exchanging communications.

27. The method of claim 1 further comprising the step of creating notifications to be sent to the user regarding the stated goal

28. The method of claim 1 further comprising an Empower Feature library.

29. The method of claim 1 further comprising the step of prompting the user to say a prayer about the stated goal.
